(19) United States
(12) Patent Application Publication
(10) Pub. No.: US 2007/0142460 A1
(43) Pub. Date: Jun. 21, 2007

(54) Compounds, compositions and methods

(76) Inventors: Cynthia A. Parrish, Collegeville, PA (US); Dashyant Dhanak, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/583,166

(22) PCT Filed: Dec. 17, 2004
(86) PCT No.: PCT/US04/42890

§ 371 Date: Jun. 16, 2006

Related U.S. Application Data
(60) Provisional application No. 60/531,568, filed on Dec. 19, 2003.

(51) Int. Cl. A61K 31/36; C07D 311/78; C07D 317/44; A61K 31/277; A61K 31/16

(52) U.S. Cl. 514/452; 558/406; 564/79; 514/522; 514/600; 514/464; 549/382; 549/440

(57) ABSTRACT

Compounds useful for treating cellular proliferative diseases and disorders by modulating the activity of KSP are disclosed.

### FIELD OF THE INVENTION

This invention relates to compounds which are inhibitors of the mitotic kinesin KSP and are useful in the treatment of cellular proliferative diseases, for example cancer, hyperplasias, restenosis, cardiac hypertrophy, immune disorders, fungal disorders, and inflammation.

### BACKGROUND OF THE INVENTION

Among the therapeutic agents used to treat cancer are the taxanes and vinca alkaloids, which act on microtubules. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Among the mitotic kinesins which have been identified is KSP. KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human, organisms, bundle antiparallel microtubules and slide them relative to one another, thus forcing the two spindle poles apart. KSP may also mediate in anaphase B spindle elongation and focussing of microtubules at the spindle pole.

Human KSP (also termed HsEg5) has been described (Blangy, et al., Cell, 83:1159-69 (1995); Whitehead, et al., Arthritis Rheum., 39:1635-42 (1996); Galgio et al., J. Cell Biol., 135:339-414 (1996); Blangy, et al., J Biol. Chem., 272:19418-24 (1997); Blangy, et al., Cell Motil Cytoskeleton, 40:174-82 (1998); Whitehead and Rattner, J. Cell Sci., 111:2551-61 (1998); Kaiser, et al., JBC 274:18925-31 (1999); GenBank accession numbers: X85137, NM004523 and U37426), and a fragment of the KSP gene (TRIP5) has been described (Lee, et al., Mol Endocrinol., 9:243-54 (1995); GenBank accession number L40372). Xenopus KSP homologs (Eg5), as well as Drosophila KLP61 F/KRP130 have been reported.

Mitotic kinesins are attractive targets for the discovery and development of novel anti-mitotic chemotherapeutics. Accordingly, it is an object of the present invention to provide compounds, compositions and methods useful in the inhibition of KSP, a mitotic kinesin.

### SUMMARY OF THE INVENTION

In accordance with the objects outlined above, the present invention provides compounds, compositions and methods that can be used to treat diseases of proliferating cells. The compounds are KSP inhibitors, particularly human KSP inhibitors.

In one aspect, the invention relates to methods for treating cellular proliferative diseases, for treating disorders by modulating the activity of KSP, and for inhibiting KSP kinesin. The methods employ compounds represented by Formula I: 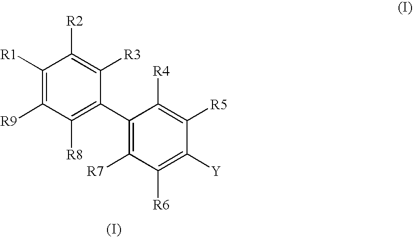
wherein:
one of R₁ and R² is —NR17S(O)ₘNR10R11 and the other is selected from the group consisting of hydrogen, halogen, hydroxy, optionally substituted lower alkoxy (including optionally substituted lower haloalkoxy), cyano, nitro, optionally substituted lower alkyl (including optionally substituted lower haloalkyl), optionally substituted heterocycloalkyl, optionally substituted aryl, optionally substituted heteroaryl, —NR13R14, optionally substituted aminosulfonyl, and optionally substituted aminocarbonyl;

R10 and R11 are independently selected from the group consisting of hydrogen, hydroxy, optionally substituted lower alkoxy, optionally substituted lower alkyl (including optionally substituted lower haloalkyl, optionally substituted aralkyl, optionally substituted heteroaralkyl, optionally substituted aryl, and optionally substituted heteroaryl;

R17 is selected from hydrogen and lower alkyl;

m is 1 or 2;

R3, R4, R5, R6, R7, R8, and R9 are independently selected from the group consisting of hydrogen, halogen, hydroxy, optionally substituted lower alkoxy (including optionally substituted lower haloalkoxy), cyano, nitro, optionally substituted lower alkyl (including optionally substituted lower haloalkyl), optionally substituted heterocycloalkyl, optionally substituted aryl, optionally substituted heteroaryl, —NR13R14, optionally substituted aminosulfonyl, and optionally substituted aminocarbonyl;

Y is selected from the group consisting of optionally substituted lower alkyl (including optionally substituted haloalkyl), halogen, trifluoromethoxy, —S(O)ₙCF₃, —CR15R16CF3, and —C(X)CF₃,

X is selected from the group consisting of oxygen and sulfur;

R15 and R16 taken together with the carbon to which they are attached form a saturated or unsaturated ring having 3 to 6 carbon atoms, optionally containing 1, 2, or 3 heteroatoms selected from nitrogen, sulfur, and oxygen, which ring is optionally substituted with halogen, hydroxy, optionally substituted lower alkoxy, cyano, or optionally substituted lower alkyl (including optionally substituted lower haloalkyl;

n is 0, 1, or 2;

or Y and R5, or Y and R6, taken together with the carbons to which they are attached form a 5 to 7 membered saturated or unsaturated ring optionally containing 1 to 2 heteroatoms selected from oxygen, nitrogen and sulfur, which ring is optionally substituted with halogen, hydroxy, cyano, —NR13R14, optionally substituted lower alkyl (including optionally substituted lower haloalkyl), or optionally substituted lower alkoxy, which ring may be aromatic or non-aromatic;

R13 and R14 are independently selected from the group consisting of hydrogen, hydroxy, optionally substituted lower alkoxy, optionally substituted lower alkyl (including optionally substituted lower haloalkyl), optionally substituted aryl, and optionally substituted heteroaryl, or R13 and R14 taken together with the nitrogen to which they are attached form a ring having 3 to 7 carbon atoms, optionally containing 1, 2, or 3 heteroatoms selected from nitrogen, sulfur, and oxygen, which ring is optionally substituted with halogen, hydroxy, cyano, optionally substituted lower alkyl (including optionally substituted lower haloalkyl), or optionally substituted lower alkoxy;

including single stereoisomers and mixtures of stereoisomers,

or a pharmaceutically acceptable derivative (including salts) or solvate thereof.

In one aspect, the invention relates to methods for treating cellular proliferative diseases and other disorders that can be treated by modulating KSP kinesin activity and for inhibiting KSP by the administration of a therapeutically effective amount of a compound of Formula I or a pharmaceutically acceptable derivative or solvate thereof. Diseases and disorders that respond to therapy with compounds of the invention include cancer, hyperplasia, restenosis, cardiac hypertrophy, immune disorders, fungal disorders and inflammation.

In another aspect, the invention relates to compounds useful in inhibiting KSP kinesin. The compounds have the structures represented by Formula I, or a derivative or solvate thereof. The invention also relates to a pharmaceutical composition containing a therapeutically effective amount of a compound of Formula I or a pharmaceutically acceptable derivative or solvate thereof admixed with at least one pharmaceutically acceptable excipient.

In an additional aspect, the present invention provides methods of screening for compounds that will bind to a KSP kinesin, for example compounds that will displace or compete with the binding of the compounds of the invention. The methods comprise combining a labeled compound of the invention, a KSP kinesin, and at least one candidate agent and determining the binding of the candidate bioactive agent to the KSP kinesin.

In a further aspect, the invention provides methods of screening for modulators of KSP kinesin activity. The methods comprise combining a compound of the invention, a KSP kinesin, and at least one candidate agent and determining the effect of the candidate bioactive agent on the KSP kinesin activity.

### BRIEF DESCRIPTION OF THE DRAWINGS

FIG. 1 shows representative compounds of the invention.

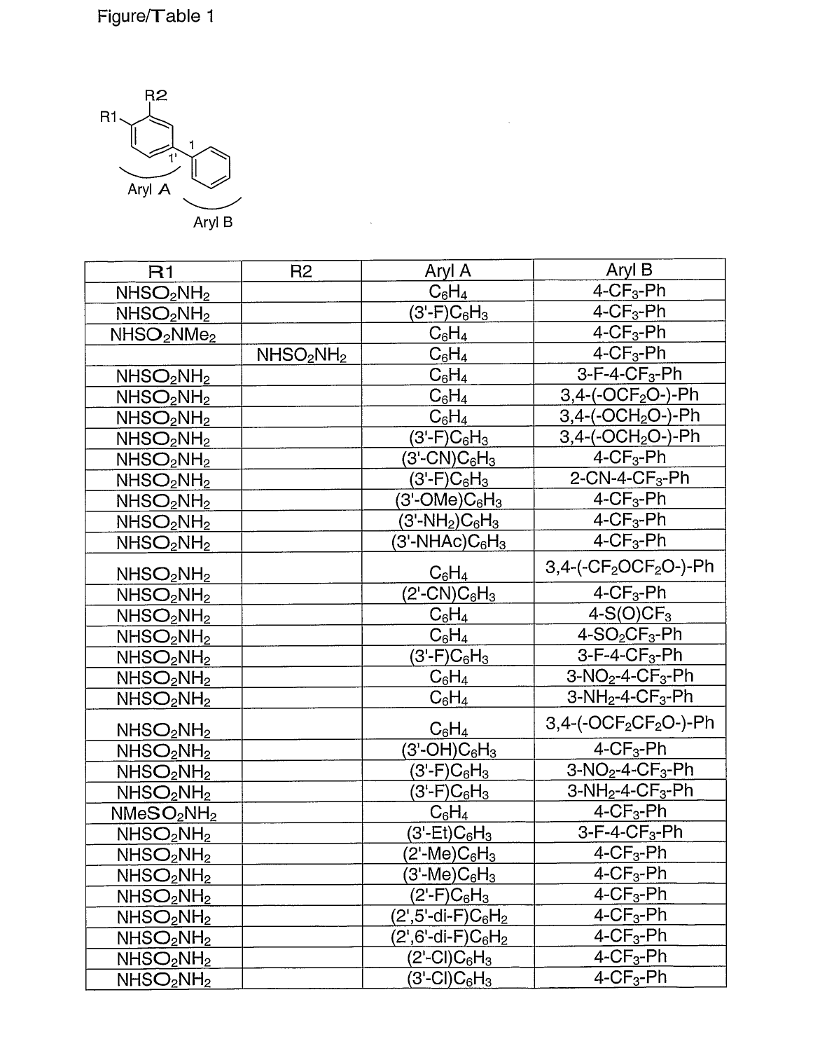

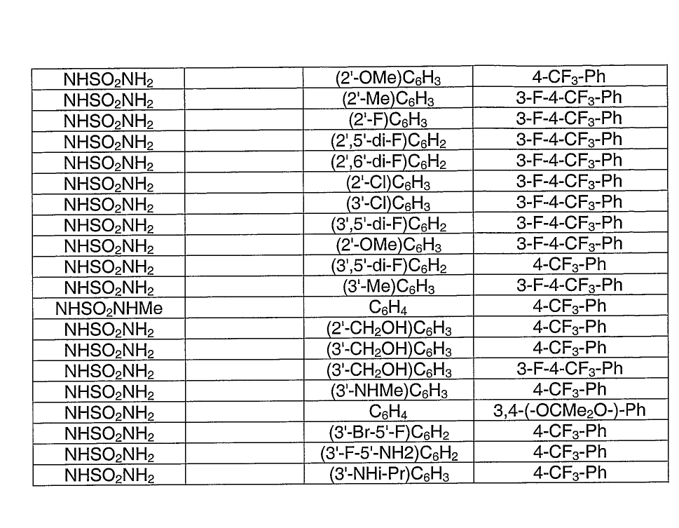

### DETAILED DESCRIPTION OF THE INVENTION

Definitions

Alkyl is intended to include linear, branched, or cyclic hydrocarbon structures and combinations thereof. Lower alkyl refers to alkyl groups of from one to five, particularly one to four (e.g., one to three), carbon atoms. Examples of lower alkyl groups include methyl, ethyl, propyl, isopropyl, butyl, s-and t-butyl and the like. Preferred alkyl groups are those of C₂₀ or below. More preferred alkyl groups are those of C₁₃ or below. Cycloalkyl is a subset of alkyl and includes cyclic hydrocarbon groups of from 3 to 13 carbon atoms. Examples of cycloalkyl groups include c-propyl, c-butyl, c-pentyl, norbornyl, adamantyl and the like. In this application, alkyl refers to alkanyl, alkenyl and alkynyl residues; it is intended to include cyclohexylmethyl, vinyl, allyl, isoprenyl and the like. Alkylene is another subset of alkyl, referring to the same residues as alkyl, but having two points of attachment. Examples of alkylene include ethylene (—CH₂CH₂—), propylene (—CH₂CH₂CH₂—), dimethylpropylene (—CH₂C(CH₃) ₂CH₂—) and cyclohexylpropylene (—CH₂CH₂CH(C₆H₁₃)—). When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl.

Alkoxy or alkoxyl refers to the group —O-alkyl, preferably including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to five, particularly one to four (e.g., one to three), carbons.

Antimitotic refers to a drug for inhibiting or preventing mitosis, for example, by causing metaphase arrest. Some antitumour drugs block proliferation and are considered antimitotics.

Aryl and heteroaryl mean a 6-membered aromatic or a 5- or 6-membered heteroaromatic ring containing 0 or 1-4 heteroatoms, respectively, selected from O, N, or S; a bicyclic 9- or 10-membered aromatic or heteroaromatic ring system containing 0 or 1-4 (or more) heteroatoms, respectively, selected from O, N, or S; or a tricyclic 12- to 14-membered aromatic or heteroaromatic ring system containing 0 or 1-4 (or more) heteroatoms, respectively, selected from O, N, or S. The aromatic 6- to 14-membered carbocyclic rings include, e.g., phenyl, naphthyl, indanyl, tetralinyl, and fluorenyl and the 5- to 10-membered aromatic heterocyclic rings include, e.g., imidazolyl, pyridinyl, indolyl, thienyl, benzopyranonyl, thiazolyl, furanyl, benzimidazolyl, quinolinyl, isoquinolinyl, quinoxalinyl, pyrimidinyl, pyrazinyl, tetrazolyl and pyrazolyl.

Aralkyl refers to a residue in which an aryl moiety is attached to the parent structure via an alkyl residue. Examples include benzyl, phenethyl, phenylvinyl, phenylallyl and the like. Heteroaralkyl refers to a residue in which a heteroaryl moiety is attached to the parent structure via an alkyl residue. Examples include furanylmethyl, pyridinylmethyl, pyrimidinylethyl and the like.

Aralkoxy refers to the group aralkyl-O—. Similarly, heteroaralkoxy refers to the group heteroaralkyl-O—; aryloxy refers to the group —O-aryl; and acyloxy refers to the group —O-acyl.

Aminocarbonyl refers to the group —NR^{b}COR^{a} or NR^{b}CO₂R^{a}, where

R^{a} is an optionally substituted C₁-C₆ alkyl, aryl, heteroaryl, aryl-C₁-C₄ alkyl-, or heteroaryl-C₁-C₄ alkyl- group, or, together with the carbon to which it is attached, form a ring having 3-6 carbon atoms, optionally containing 1, 2 or 3 heteroatoms selected from nitrogen, sulfur and oxygen, which ring is optionally substituted with halogen, hydroxy, optionally substituted alkoxy, cyano, optionally substituted lower alkyl, or optionally substituted lower haloalkyl; and

R^{b} is H or optionally substituted C₁-C₆ alkyl-, aryl-, heteroaryl-, aryl-C₁-C₄ alkyl-, or heteroaryl-C₁-C₄ alkyl- group; and

where each optionally substituted Ra and Rb group is independently unsubstituted or substituted with one or more substituents independently selected from C₁-C₄ alkyl-, aryl-, heteroaryl-, aryl-C₁-C₄ alkyl-, heteroaryl-C₁-C₄ alkyl-, C₁-C₄ haloalkyl-, —OC₁-C₄ alkyl-, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Halogen or halo refers to fluorine, chlorine, bromine or iodine. Fluorine, chlorine and bromine are preferred. Haloalkyl and haloalkoxy refer to alkyl and alkoxy, respectively, wherein the alkyl moiety is substituted with one or more halogens which may be the same or different. Dihaloaryl, dihaloalkyl, trihaloaryl etc. refer to aryl and alkyl substituted with a plurality of halogens, but not necessarily a plurality of the same halogen; thus 4-chloro-3-fluorophenyl is within the scope of dihaloaryl.

Heterocycloalkyl refers to a saturated or unsaturated monocyclic ring having from 5 to 7 member atoms and containing from 1 to 3 heteroatoms as member atoms in the ring. Heterocycloalkyl rings are not aromatic. Heterocycloalkyl groups containing more than one heteroatom may contain different heteroatoms. Heterocycloalkyl groups may be optionally substituted with one or more substituents as defined herein. In certain embodiments, heterocycloalkyl is saturated. In other embodiments, heterocycloalkyl is unsaturated but not aromatic. Heterocycloalkyl includes pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, piperazinyl, morpholinyl, thiamorpholinyl, azepinyl, 1,3-dioxolanyl, 1,3-dioxanyl, 1,4-dioxanyl, 1,3-oxathiolanyl, 1,3-oxathianyl, 1,3-dithianyl.

It will be understood that “optional” or optionally” means that the subsequently described event or circumstance may or may not occur, and that the description includes instances where said event or circumstance occurs and instances in which it does not. It will be understood that when a group or moiety is “optionally substituted,” the group or moiety may be unsubstituted or may be substituted by one or more of the substituents defined herein, where each substituent is selected independently. It will be further understood by those skilled in the art with respect to any groups containing one or more substituents that such groups are not intended to introduce any substituent or substitution patterns that are sterically impractical and/or synthetically non-feasible and/or inherently unstable.

Solvate refers to the compound formed by the interaction of a solvent and a compound of Formula I or a pharmaceutically acceptable derivative thereof. Suitable solvates are those formed with pharmaceutically acceptable solvents, including hydrates (i.e., wherein the solvent is water). It will be understood that phrases such as “a compound of Formula I or a pharmaceutically acceptable derivative (e.g., salt) or solvate thereof” are intended to encompass the compound of Formula I, a pharmaceutically acceptable derivative (e.g., salt) of the compound, a solvate of the compound and a solvate of a pharmaceutically acceptable derivative (e.g., salt) of the compound.

Substituted alkoxy refers to the group —O-(substituted alkyl).

Substituted-alkyl, heterocycloalkyl, aryl, and heteroaryl, which includes the substituted alkyl, heterocycloalkyl, aryl and heteroaryl moieties of any group containing an optionally substituted alkyl, heterocycloalkyl, aryl or heteroaryl moiety (e.g., alkoxy, aralkyl or heteroaralkyl), refer respectively to alkyl, heterocycloalkyl, aryl, and heteroaryl wherein one or more (e.g., up to or about 5; in one embodiment up to or about 3) hydrogen atoms are replaced by a substituent, wherein each substituent is independently selected from the group:

—R^{a}, —OR^{b}, —O(C₁-C₂ alkyl)O— (as an aryl substituent), —SR^{b}, —NR^{b}R^{c}, —C(═NR^{c})—NR^{b}R^{c}, halogen, cyano, nitro, —COR^{b}, —CO₂R^{b}, —CONR^{b}R^{c}, —OCOR^{b}, —OCO₂R^{b}, —OCONR^{b}R^{c}, —NR^{c}COR^{b}, —NR^{c}CO₂R^{b}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a},

where R^{a} is an optionally substituted C₁-C₆ alkyl, aryl, heteroaryl, aryl-C₁-C₄ alkyl-, or heteroaryl-C₁-C₄ alkyl- group, or, together with the carbon to which it is attached, form a ring having 3-6 carbon atoms, optionally containing 1, 2 or 3 heteroatoms selected from nitrogen, sulfur and oxygen, which ring is optionally substituted with halogen, hydroxy, optionally substituted alkoxy, cyano, optionally substituted lower alkyl, or optionally substituted lower haloalkyl;

R^{b} is H or optionally substituted C₁-C₆ alkyl, aryl, heteroaryl, aryl-C₁-C₄ alkyl-, or heteroaryl-C₁-C₄ alkyl- group;

R^{c} is hydrogen or C₁-C₄ alkyl;

where each optionally substituted R^{a} group and R^{b} group is independently unsubstituted or substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO2(C₁-C₄ alkyl), —SO₂(phenyl), —S₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Substituted haloalkyl and haloalkoxy refer to alkyl and alkoxy, respectively, wherein in addition to the halogen(s) substituent(s), one or more (e.g., up to or about 5; in one embodiment up to or about 3) hydrogen atoms are replaced by a substituent, wherein each substituent is independently selected from the group:

—R^{a}, —OR^{b}, —O(C₁-C₂ alkyl)O— (as an aryl substituent), —SR^{b}, —C(═NR^{c})—NR^{b}R^{c}, cyano, nitro, —COR^{b}, —CO₂R^{b}, —CONR^{b}R^{c}, —OCOR^{b}, —OCO₂R^{b}, —OCONR^{b}R^{c}, —NR^{c}COR^{b}, —NR^{c}CO₂R^{b}, —NR^{c}CONR^{b}R^{c}, —CO₂R^{b}, —CONR^{b}R^{c}, —NR^{c}COR^{b}, —SOR^{a}, —SO₂R^{a}, —SO₂NR^{b}R^{c}, and —NR^{c}SO₂R^{a},

where R^{a} is an optionally substituted C₁-C₆ alkyl, aryl, heteroaryl, aryl-C₁-C₄ alkyl-, or heteroaryl-C₁-C₄ alkyl- group, or, together with the carbon to which it is attached, form a ring having 3-6 carbon atoms, optionally containing 1, 2 or 3 heteroatoms selected from nitrogen, sulfur and oxygen, which ring is optionally substituted with halogen, hydroxy, optionally substituted alkoxy, cyano, optionally substituted lower alkyl, or optionally substituted lower haloalkyl;

R^{b} is H or optionally substituted C₁-C₆ alkyl, aryl, heteroaryl, aryl-C₁-C₄ alkyl-, or heteroaryl-C₁-C₄ alkyl- group;

R^{c} is hydrogen or C₁-C₄ alkyl;

where each optionally substituted R^{a} group and R^{b} group is independently unsubstituted or substituted with one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Aminosulfonyl refers to the group —NR^{b}SO₂R^{a} or NR^{b}SO₃R^{a}, where

R^{a} is an optionally substituted C₁-C₆ alkyl, aryl, heteroaryl, aryl-C₁-C₄ alkyl-, or heteroaryl-C₁-C₄ alkyl- group, or, together with the carbon to which it is attached, form a ring having 3-6 carbon atoms, optionally containing 1, 2 or 3 heteroatoms selected from nitrogen, sulfur and oxygen, which ring is optionally substituted with halogen, hydroxy, optionally substituted alkoxy, cyano, optionally substituted lower alkyl, or optionally substituted lower haloalkyl; and

R^{b} is H or optionally substituted C₁-C₆ alkyl-, aryl-, heteroaryl-, aryl-C₁-C₄ alkyl-, or heteroaryl-C₁-C₄ alkyl- group; and

where each optionally substituted R^{a} and R^{b} group is independently unsubstituted or substituted with one or more substituents independently selected from C₁-C₄ alkyl-, aryl-, heteroaryl-, aryl-C₁-C₄ alkyl-, heteroaryl-C₁-C₄ alkyl-, C₁-C₄ haloalkyl-, —OC₁-C₄ alkyl-, —OC₁-C₄ alkylphenyl, —C₁-C₄ alkyl-OH, —OC₁-C₄ haloalkyl, halogen, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

Pharmaceutically acceptable derivatives of Formula I include any pharmaceutically acceptable salt, ester, or salt of such ester, of a compound of Formula I which, upon administration to the recipient is capable of providing (directly or indirectly) a compound of Formula I or an active metabolite or residue thereof. For simplicity, in certain instances herein reference is made specifically to salts of a compound of Formula I. It is appreciated that other pharmaceutically acceptable derivatives, such as esters, of a compound of Formula I are also suitable for use in the present invention in the manner specifically disclosed for salts, as though expressly set forth herein.

Pharmaceutically acceptable acid addition salt refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

Pharmaceutically acceptable base addition salts include those derived from inorganic bases such as sodium, potassium, lithium, ammonium, calcium, magnesium, iron, zinc, copper, manganese, aluminum salts and the like. In a particular embodiment ammonium, potassium, sodium, calcium, or magnesium salts are used. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

Compounds described herein which contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

When desired, the R- and S-isomers may be resolved by methods known to those skilled in the art, for example by formation of diastereoisomeric salts or complexes which may be separated, for example, by crystallisation; via formation of diastereoisomeric derivatives which may be separated, for example, by crystallisation, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where the desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting one enantiomer to the other by asymmetric transformation.

Compounds of the Present Invention

The present invention is directed to a class of novel compounds that are modulators, particularly inhibitors, of mitotic kinesins. By inhibiting or modulating mitotic kinesins, but not other kinesins (e.g., transport kinesins), specific inhibition of cellular proliferation is accomplished. Thus, the present invention capitalizes on the finding that perturbation of mitotic kinesin function causes malformation or dysfunction of mitotic spindles, frequently resulting in cell cycle arrest and cell death. The methods of inhibiting a human KSP kinesin comprise contacting an inhibitor of the invention with a KSP kinesin, particularly human KSP kinesins, including fragments and variants of KSP. The inhibition can be of the ATP hydrolysis activity of the KSP kinesin and/or the mitotic spindle formation activity, such that the mitotic spindles are disrupted. Meiotic spindles may also be disrupted.

An object of the present invention is to develop inhibitors and modulators of mitotic kinesins, in particular KSP, for the treatment of disorders associated with cell proliferation. Traditionally, dramatic improvements in the treatment of cancer, one type of cell proliferative disorder, have been associated with identification of therapeutic agents acting through novel mechanisms. Examples of this include not only the taxane class of agents that appear to act on microtubule formation, but also the camptothecin class of topoisomerase I inhibitors. The compounds, compositions and methods described herein can differ in their selectivity and are preferably used to treat diseases of proliferating cells, including, but not limited to cancer, hyperplasias, restenosis, cardiac hypertrophy, immune disorders, fungal disorders and inflammation.

Accordingly, the present invention relates to methods employing compounds represented by Formula I: 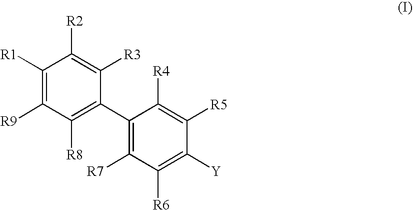
wherein:
one of R1 and R2 is —NR17S(O)mNR10R11 and the other is selected from the group consisting of hydrogen, halogen, hydroxy, optionally substituted lower alkoxy (including optionally substituted lower haloalkoxy), cyano, nitro, optionally substituted lower alkyl (including optionally substituted lower haloalkyl), optionally substituted heterocycloalkyl, optionally substituted aryl, optionally substituted heteroaryl, —NR13R14, optionally substituted aminosulfonyl, and optionally substituted aminocarbonyl;

R10 and R11 are independently selected from the group consisting of hydrogen, hydroxy, optionally substituted lower alkoxy, optionally substituted lower alkyl (including optionally substituted lower haloalkyl), optionally substituted aralkyl, optionally substituted heteroaralkyl, optionally substituted aryl, and optionally substituted heteroaryl;

R17 is selected from hydrogen and lower alkyl;

m is 1 or 2;

R3, R4, R5, R6, R7, R8, and R9 are independently selected from the group consisting of hydrogen, halogen, hydroxy, optionally substituted lower alkoxy (including optionally substituted lower haloalkoxy), cyano, nitro, optionally substituted lower alkyl (including optionally substituted lower haloalkyl), optionally substituted heterocycloalkyl, optionally substituted aryl, optionally substituted heteroaryl, —NR13R14, optionally substituted aminosulfonyl, and optionally substituted aminocarbonyl;

Y is selected from the group consisting of optionally substituted lower alkyl (including optionally substituted haloalkyl), halogen, trifluoromethoxy, —S(O)ₙCF₃, —CR15R16CF₃ an —C(X)CF₃,

X is selected from the group consisting of oxygen and sulfur;

R15 and R16 taken together with the carbon to which they are attached form a saturated or unsaturated ring having 3 to 6 carbon atoms, optionally containing 1, 2, or 3 heteroatoms selected from nitrogen, sulfur, and oxygen, which ring is optionally substituted with halogen, hydroxy, optionally substituted lower alkoxy, cyano, or optionally substituted lower alkyl (including optionally substituted lower haloalkyl);

n is 0, 1, or 2;

or Y and R5, or Y and R6, taken together with the carbons to which they are attached form a 5 to 7 membered saturated or unsaturated ring optionally containing 1 to 2 heteroatoms selected from oxygen, nitrogen and sulfur, which ring is optionally substituted with halogen, hydroxy, cyano, —NR13R14, optionally substituted lower alkyl (including optionally substituted lower haloalkyl), or optionally substituted lower alkoxy, which ring may be aromatic or non-aromatic;

R13 and R14 are independently selected from the group consisting of hydrogen, hydroxy, optionally substituted lower alkoxy, optionally substituted lower alkyl (including optionally substituted lower haloalkyl), optionally substituted aryl, and optionally substituted heteroaryl, or R13 and R14 taken together with the nitrogen to which they are attached form a ring having 3 to 7 carbon atoms, optionally containing 1, 2, or 3 heteroatoms selected from nitrogen, sulfur, and oxygen, which ring is optionally substituted with halogen, hydroxy, cyano, optionally substituted lower alkyl (including optionally substituted lower haloalkyl), or optionally substituted lower alkoxy;

or a pharmaceutically acceptable derivative or solvate thereof.

The compounds of formula (I) are intended to include single stereoisomers and mixtures of stereoisomers of formula (I).

Nomenclature

The compounds of Formula I can be named and numbered (e.g., using ACD/Name add-in for ISIS/Draw version 6.02) as described below.

For example, the compound 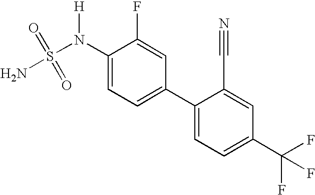
i.e., the compound of Formula I where R1 is —NHSO₂NH₂, R2 is fluoro, R3 is hydrogen, R4 is cyano, R5-R9 are hydrogen, and Y is trifluoromethyl, can be named N-[2′-cyano-3-fluoro-4′-(trifluoromethyl)-4-biphenylyl]sulfamide; 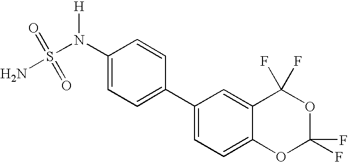
i.e., the compound of Formula I where R1 is —NHSO₂NH₂, R2-R4 and R6-R9 are hydrogen, and R5 and Y taken together with the carbons to which they are attached is 2,2,4,4-tetrafluoro-4H-1,3-dioxin, can be named N-[4-(2,2,4,4-tetrafluoro-4H-1,3-benzodioxin-6-yl)phenyl]sulfamide.

In one embodiment of the compounds of Formula I, R1 is —NR17S(O)ₘNR10R11 and R2 is selected frorn the group consisting of hydrogen, halogen, hydroxy, optionally substituted lower alkoxy (e.g., optionally substituted lower haloalkoxy), cyano, nitro, optionally substituted lower alkyl (e.g., optionally substituted lower haloalkyl), optionally substituted heterocycloalkyl, optionally substituted aryl, optionally substituted heteroaryl, —NR13R14, optionally substituted aminosulfonyl, and optionally substituted aminocarbonyl. In a particular embodiment, R1 is —NHS(O)₂NR10R11.

In another embodiment, R2 is —NR17S(O)ₘNR10R11 and R1 is selected from the group consisting of hydrogen, halogen, hydroxy, optionally substituted lower alkoxy (e.g., optionally substituted lower haloalkoxy), cyano, nitro, optionally substituted lower alkyl (e.g., optionally substituted lower haloalkyl), optionally substituted heterocycloalkyl, optionally substituted aryl, optionally substituted heteroaryl, —NR13R14, optionally substituted aminosulfonyl, and optionally substituted aminocarbonyl. In a particular embodiment, R2 is —NHS(O)₂NR10R11.

In one embodiment of the compounds of formula (I), m is 2.

In another embodiment of the compounds of Formula I, R17 is hydrogen.

In another embodiment of the compounds of formula (I), R10 and R11 are independently selected from hydrogen, hydroxy, and optionally substituted lower alkyl, or from hydrogen, hydroxy, and lower alkyl. In a further particular embodiment, R10 and R11 are independently selected from hydrogen and lower alkyl (e.g., C₁₋₃ alkyl), e.g., hydrogen and methyl. More particularly, R10 and R11 are hydrogen.

In one embodiment wherein R1 is —NR17S(O)ₘNR10R11, R2 is selected from the group consisting of hydrogen, halogen, hydroxy, optionally substituted lower alkoxy, cyano, nitro, optionally substituted lower alkyl, and —NR13R14, especially hydrogen, halogen, hydroxy, lower alkoxy, lower alkyl and —NR13R14. In particular such embodiments R2 is selected from hydrogen, halogen, hydroxy, methoxy, methyl, cyano and —NH₂, more particularly from hydrogen, halogen and —NH₂, even more particularly from hydrogen, fluoro and —NH₂, and most particularly from hydrogen and fluoro.

In one embodiment R3, R4, R7 and R8 are independently selected from the group consisting of hydrogen, halogen, hydroxy, optionally substituted lower alkoxy, cyano, nitro, optionally substituted lower alkyl, and —NR13R14, especially hydrogen, halogen, lower alkoxy, cyano, lower alkyl and —NR13R14. In particular embodiments R3, R4, R7 and R8 are independently selected from the group consisting of hydrogen, halogen, cyano, methoxy, methyl and —NH₂, more particularly hydrogen and halogen, even more particularly hydrogen and fluoro. In a particular embodiment each of R3, R4, R7 and R8 are hydrogen.

In one embodiment, R9 is selected from the group consisting of hydrogen, halogen, hydroxy, optionally substituted lower alkoxy, cyano, nitro, optionally substituted lower alkyl, and —NR13R14, especially hydrogen, halogen, lower alkyl and —NR13R14. In particular embodiments Rg is selected from hydrogen, halogen, methyl and —NH₂, more particularly hydrogen and halogen, even more particularly hydrogen and fluoro. In a particular embodiment R9 is hydrogen.

In one embodiment of compounds of formula (I), Y is halogen. In particular embodiments, Y is selected from fluoro, chloro, and bromo, more particularly chloro and bromo, most particularly bromo.

In another embodiment, Y is trifluoromethoxy.

In another embodiment, Y is S(O)ₙCF₃, particularly S(O)₂CF₃ or S(O)CF₃, more particularly S(O)₂CF₃.

In another embodiment, Y is optionally substituted lower alkyl. In particular embodiments Y is selected from butyl (e.g., isobutyl, t-butyl), and propyl (e.g., isopropyl), more particularly isopropyl and t-butyl. In another particular embodiment Y is optionally substituted lower haloalkyl, particularly trifluoromethyl. In another particular embodiment, Y is —CR15R16CF₃, e.g., 3-trifluoromethyl-diazirin-3-yl.

In another embodiment of the compounds of formula (I), Y and R5, or Y and R6, taken together with the carbons to which they are attached form an optionally substituted 5 or 6 membered saturated or unsaturated ring containing 1 to 2 (especially 2) heteroatoms selected from oxygen, nitrogen and sulfur (especially oxygen or sulfur, more especially oxygen), which ring may be aromatic or non-aromatic. In a particular embodiment the ring is substituted with one or more halogen, lower alkyl, and/or lower haloalkyl groups, particularly fluoro, methyl and/or trifluorornethyl.

In a particular embodiment, Y and R5, or Y and R6, taken together with the carbons to which they are attached, form an optionally substituted dioxole or dioxin. In a particular embodiment, Y and R5, or Y and R6, taken together with the carbons to which they are attached, form 2,2-difluoro-1,3-dioxole or 2,2,4,4-tetrafluoro-4H-1,3-dioxin.

In one embodiment of compounds of formula (I), R5 and R6 are independently selected from the group consisting of hydrogen, halogen, hydroxy, optionally substituted lower alkoxy, cyano, nitro, optionally substituted lower alkyl, and —NR13R14, especially hydrogen, halogen, nitro, optionally substituted lower alkyl and —NR13R14. In particular embodiments R5 and R6 are independently selected from hydrogen, halogen, methyl, nitro, trifluoromethyl, and —NH₂, more particularly hydrogen, halogen and trifluoromethyl, most particularly hydrogen and fluoro.

Particular examples of compounds of the present invention include:
N-[4′-(trifluoromethyl)-4-biphenylyl]sulfamide
N-[3-fluoro-4′-(trifluoromethyl)-4-biphenylyl]sulfamide
N,N-dimethyl-N′-[4′-(trifluoromethyl)-4-biphenylyl]sulfamide
N-[4′-(trifluoromethyl)-3-biphenylyl]sulfamide
N-[3′-fluoro-4′-(trifluoromethyl)-4-biphenylyl]sulfamide
N-[4-(2,2-difluoro-1,3-benzodioxol-5-yl)phenyl]sulfamide
N-[4-(1,3-benzodioxol-5-yl)phenyl]sulfamide
N-[4-(1,3-benzodioxol-5-yl)-2-fluorophenyl]sulfamide
N-[3-cyano-4′-(trifluoromethyl)-4-biphenylyl]sulfamide
N-[2′-cyano-3-fluoro-4′-(trifluoromethyl)-4-biphenylyl]sulfamide
N-[3-(methoxy)-4′-(trifluoromethyl)-4-biphenylyl]sulfamide
N-[3-amino-4′-(trifluoromethyl)-4-biphenylyl]sulfamide
N-[4-[(aminosulfonyl)amino]-4′-(trifluoromethyl)-3-biphenylyl]acetamide
N-[4-(2,2,4,4-tetrafluoro-4H-1,3-benzodioxin-6-yl)phenyl]sulfamide
N-[2-cyano-4′-(trifluoromethyl)-4-biphenylyl]sulfamide
N-{4′-[(trifluoromethyl)sulfinyl]-4-biphenylyl}sulfamide
N-{4′-[(trifluoromethyl)sulfonyl]-4-biphenylyl}sulfamide
N-[3,3′-difluoro-4′-(trifluoromethyl)-4-biphenylyl]sulfamide
N-[3′-nitro-4′-(trifluoromethyl)-4-biphenylyl]sulfamide
N-[3′-amino-4′-(trifluoromethyl)-4-biphenylyl]sulfamide
N-[4-(2,2,3,3-tetrafluoro-2,3-dihydro-1,4-benzodioxin-6-yl)phenyl]sulfamide
N-[3-hydroxy-4′-(trifluoromethyl)-4-biphenylyl]sulfamide
N-[3-fluoro-3′-nitro-4′-(trifluoromethyl)-4-biphenylyl]sulfamide
N-[3′-amino-3-fluoro-4′-(trifluoromethyl)-4-biphenylyl]sulfamide
N-methyl-N-[4′-(trifluoromethyl)-4-biphenylyl]sulfamide
N-[3-ethyl-3′-fluoro-4′-(trifluoromethyl)-4-biphenylyl]sulfamide
N-[2-methyl-4′-(trifluoromethyl)-4-biphenylyl]sulfamide
N-[3-methyl-4′-(trifluoromethyl)-4-biphenylyl]sulfamide
N-[2-fluoro-4′-(trifluoromethyl)-4-biphenylyl]sulfamide
N-[2,5-difluoro-4′-(trifluoromethyl)-4-biphenylyl]sulfamide
N-[2,6-difluoro-4′-(trifluoromethyl)-4-biphenylyl]sulfamide
N-[2-chloro-4′-(trifluoromethyl)-4-biphenylyl]sulfamide
N-[3-chloro-4′-(trifluoromethyl)-4-biphenylyl]sulfamide
N-[2-(methyloxy)-4′-(trifluoromethyl)-4-biphenylyl]sulfamide
N-[3′-fluoro-2-methyl-4′-(trifluoromethyl)-4-biphenylyl]sulfamide
N-[2,3′-difluoro-4′-(trifluoromethyl)-4-biphenylyl]sulfamide
N-[2,3′,5-trifluoro-4′-(trifluoromethyl)-4-biphenylyl]sulfamide
N-[2,3′,6-trifluoro-4′-(trifluoromethyl)-4-biphenylyl]sulfamide
N-[2-chloro-3′-fluoro-4′-(trifluoromethyl)-4-biphenylyl]sulfamide
N-[3-chloro-3′-fluoro-4′-(trifluoromethyl)-4-biphenylyl]sulfamide
N-[3,3′,5-trifluoro-4′-(trifIuoromethyl)-4-biphenylyl]sulfamide
N-[3′-fluoro-2-(methyloxy)-4′-(trifluoromethyl)-4-biphenylyl]sulfamide
N-[3,5-difluoro-4′-(trifluoromethyl)-4-biphenylyl]sulfamide
N-[3′-fluoro-3-methyl-4′-(trifluoromethyl)-4-biphenylyl]sulfamide
N-methyl-N′-[4′-(trifluoromethyl)-4-biphenylyl]sulfamide
N-[2-(hydroxymethyl)-4′-(trifluoromethyl)-4-biphenylyl]sulfamide
N-[3-(hydroxymethyl)-4′-(trifluoromethyl)-4-biphenylyl]sulfamide
N-[3′-fluoro-3-(hydroxymethyl)-4′-(trifluoromethyl)-4-biphenylyl]sulfamide
N-[3-(methylamino)-4′-(trifluoromethyl)-4-biphenylyl]sulfamide
N-[4-(2,2-dimethyl-1,3-benzodioxol-5-yl)phenyl]sulfamide
N-[3-bromo-5-fluoro-4′-(trifluoromethyl)-4-biphenylyl]sulfamide
N-[3-amino-5-fluoro-4′-(trifluoromethyl)-4-biphenylyl]sulfamide and
N-[3-[(1-methylethyl)amino]-4′-(trifluoromethyl)-4-biphenylyl]sulfamide.

The compounds of the invention may be prepared as shown in the General Methods and as described below, utilizing techniques well known in the art. The starting materials for the schemes shown in the figures are commercially available, e.g., from Aldrich Chemical Company, Milwaukee, Wis. or may be readily prepared by those skilled in the art using commonly employed synthetic methodology.

If an inventive compound is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; such as ethylenediamine, and cyclic amines, such as cyclohexylamine, piperidine, morpholine, and piperazine; as well as inorganic salts derived from sodium, calcium, potassium, magnesium, manganese, iron, copper, zinc, aluminum, and lithium.

If an inventive compound is a base, a desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, or the like.

Once made, the compounds of the invention find use in a variety of applications. As will be appreciated by those skilled in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In a preferred embodiment, the compounds of the invention are used to modulate mitotic spindle formation, thus causing prolonged cell cycle arrest in mitosis. By “modulate” herein is meant altering mitotic spindle formation, including increasing and decreasing spindle formation. By “mitotic spindle formation” herein is meant organization of microtubules into bipolar structures by mitotic kinesins. By “mitotic spindle dysfunction” herein is meant mitotic arrest and monopolar spindle formation.

The compounds of the invention are useful to bind to and/or modulate the activity of a mitotic kinesin, KSP. In a preferred embodiment, the KSP is human KSP, although KSP kinesins from other organisms may also be used. In this context, modulate means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP. See U.S. Pat. Nos. 6,414,121 and 6,437,115, hereby incorporated by reference in their entirety. In addition, other mitotic kinesins may be used in the present invention. However, the compounds of the invention have been shown to have specificity for KSP.

For assay of activity, generally either KSP or a compound according to the invention is non-diffusably bound to an insoluble support having isolated sample receiving areas (e.g., a microtiter plate, an array, etc.). The insoluble support may be made of any composition to which the compounds can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the compound is not crucial so long as it is compatible with the reagents and overall methods of the invention, maintains the activity of the compound and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

The anti-mitotic agents of the invention may be used on their own to modulate the activity of a mitotic kinesin, particularly KSP. In this embodiment, the anti-mitotic agents of the invention are combined with KSP and the activity of KSP is assayed. Kinesin activity is known in the art and includes one or more kinesin activities. Kinesin activities include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes; such as kinases or proteases; and specific kinesin cellular activities such as spindle pole separation.

Methods of performing motility assays are well known to those of skill in the art. (See e.g., Hall, et al. (1996), Biophys. J., 71: 3467-3476, Turner et al., 1996, AnaL Biochem. 242 (1):20-5; Gittes et al., 1996, Biophys. J. 70(I): 418-29; Shirakawa et al., 1995, J. Exp. BioL 198: 1809-15; Winkelmann et al., 1995, Biophys. J. 68: 2444-53; Winkelmann et al., 1995, Biophys. J. 68: 72S.)

Methods known in the art for determining ATPase hydrolysis activity also can be used. Preferably, solution based assays are utilized. U.S. patent application Ser No. 09/314,464, filed May 18,1999 (U.S. Pat. No. 6,410,254), hereby incorporated by reference in its entirety, describes such assays. Alternatively, conventional methods are used. For example, Pᵢ release from kinesin can be quantified. In one preferred embodiment, the ATPase hydrolysis activity assay utilizes 0.3 M PCA (perchloric acid) and malachite green reagent (8.27 mM sodium molybdate II, 0.33 mM malachite green oxalate, and 0.8 mM Triton X-100). To perform the assay, 10 μL of reaction is quenched in 90 μL of cold 0.3 M PCA. Phosphate standards are used so data can be converted to mM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to mM Pᵢ and plotted over time. Additionally, ATPase assays known in the art include the luciferase assay.

ATPase activity of kinesin motor domains also can be used to monitor the effects of modulating agents. In one embodiment ATPase assays of kinesin are performed in the absence of microtubules. In another embodiment, the ATPase assays are performed in the presence of microtubules.

Different types of modulating agents can be detected in the above assays. In one embodiment, the effect of a modulating agent is independent of the concentration of microtubules and ATP. In another embodiment, the effect of the agents on kinesin ATPase can be decreased by increasing the concentrations of ATP, microtubules or both (i.e., the effect can be increased by decreasing the concentrations of ATP, microtubules or both). In yet another embodiment, the effect of the modulating agent is increased by increasing concentrations of ATP, microtubules or both.

Agents that modulate the biochemical activity of KSP in vitro may then be screened in vivo. Methods for such agents in vivo include assays of cell cycle distribution, cell viability, or the presence, morphology, activity, distribution, or amount of mitotic spindles. Methods for monitoring cell cycle distribution of a cell population, for example, by flow cytometry, are well known to those skilled in the art, as are methods for determining cell viability. See for example, U.S. Patent Application “Methods of Screening for Modulators of Cell Proliferation and Methods of Diagnosing Cell Proliferation States,” filed Oct. 22, 1999, Ser. No. 09/428,156 (U.S. Pat. No. 6,617,115), hereby incorporated by reference in its entirety.

In addition to the assays described above, microscopic methods for monitoring spindle formation and malformation are well known to those of skill in the art (see, e.g., Whitehead and Rattner (1998), J. Cell Sci. 111:2551-61; Galgio et al, (1996) J. Cell biol., 135:399-414).

The compounds of the invention inhibit the KSP kinesin. One measure of inhibition is IC₅₀, defined as the concentration of the compound at which the activity of KSP is decreased by fifty percent relative to a control. Preferred compounds have IC₅₀'s of less than about 1 mM, with preferred embodiments having IC₅₀'s of less than about 100 μM, with more preferred embodiments having IC₅₀'s of less than about 10 μM, with particularly preferred embodiments having IC₅₀'s of less than about 1 μM, and especially preferred embodiments having IC₅₀'s of less than about 100 nM, and more preferably less than about 10 nM. Measurement of IC₅₀ is done using an ATPase assay.

Another measure of inhibition is Kᵢ. For compounds with IC₅₀'s less than 1 μM, the Kᵢ or K_{d} is defined as the dissociation rate constant for the interaction of the compounds described herein with KSP. Preferred compounds have Kᵢ's of less than about 100 μM, with preferred embodiments having Kᵢ's of less than about 10 μM, with particularly preferred embodiments having Kᵢ's of less than about 1 μM, and especially preferred embodiments having Kᵢ's of less than about 100 nM. The Kᵢ for a compound is determined from the IC₅₀ based on three assumptions. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ I - \frac{( {E}_{0} + {I}_{0} + Kd ) - \sqrt{{( {E}_{0} + {I}_{0} + Kd )}^{2} - 4 {E}_{0} {I}_{0}}}{2 {E}_{0}} ]$$

where V is the observed rate, Vₘₐₓ is the rate of the free enzyme, I₀ is the inhibitor concentration, E₀ is the enzyme concentration, and K_{d} is the dissociation constant of the enzyme-inhibitor complex.

Another measure of inhibition is GI₅₀, defined as the concentration of the compound that results in a decrease in the rate of cell growth by fifty percent. Preferred compounds have GI₅₀'s of less than about 1 mM. The level of preferability of embodiments is a function of their GI₅₀: those having GI₅₀'s of less than about 20 μM are more preferred; those having GI₅₀'s of 10 μM more so; those having GI₅₀ of less than about 1 μM more so; those having GI₅₀'s of less than about 100 nM even more so. Measurement of GI₅₀ is done using a cell proliferation assay.

The compounds, compositions and methods of the invention are used to treat cellular proliferation diseases. Disease states which can be treated by the compounds, compositions and methods provided herein include, but are not limited to, cancer (further discussed below), autoimmune disease, fungal disorders, arthritis, graft rejection, inflammatory bowel disease, proliferation induced after medical procedures, including, but not limited to, surgery, angioplasty, and the like. It is appreciated that in some cases the cells may not be in a hyper or hypo proliferation state (abnormal state) and still require treatment. Thus, in one embodiment, the invention herein includes application to cells or individuals afflicted or impending affliction with any one of these disorders or states.

The compounds, compositions and methods provided herein are particularly deemed useful for the treatment of cancer including solid tumors such as skin, breast, brain, cervical carcinomas, testicular carcinomas, etc. More particularly, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

Accordingly, the compounds of the invention are administered to cells. By “administered” herein is meant administration of a therapeutically effective dose of the anti-mitotic agents of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant any cell in which mitosis or meiosis can be altered.

A “patient” for the purposes of the present invention includes both humans and other animals, particularly mammals, and other organisms. Thus the methods are applicable to both human therapy and veterinary applications. In the preferred embodiment the patient is a mammal, and in the most preferred embodiment the patient is human.

Anti-mitotic agents having the desired pharmacological activity may be administered in a physiologically acceptable carrier to a patient, as described herein. Depending upon the manner of introduction, the compounds may be formulated in a variety of ways as discussed below. The concentration of therapeutically active compound in the formulation may vary from about 0.1-99.9 wt. %. The compounds of Formula I, and the pharmaceutically acceptable derivatives and solvates thereof can be administered alone or in combination with other treatments, i.e., radiation, or other therapeutic agents, such as the taxane class of agents that appear to act on microtubule formation or the camptothecin class of topoisomerase I inhibitors. When so-used, other therapeutic agents can be administered before, concurrently (whether in separate dosage forms or in a combined dosage form), or after administration of an active agent of the present invention.

In a preferred embodiment, the pharmaceutical compositions are in a water soluble form, such as pharmaceutically acceptable salts, which is meant to include both acid and base addition salts.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents. The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol. Additives are well known in the art, and are used in a variety of formulations.

Pharmaceutical formulations include a compound of Formula I or a pharmaceutically acceptable derivative or solvate thereof, and one or more pharmaceutically acceptable excipients. As is known in the art, pharmaceutical excipients are secondary ingredients that function to enable or enhance the delivery of a drug or medicine in a variety of dosage forms (e.g.: oral forms such as tablets, capsules, and liquids; topical forms such as dermal, opthalmic, and otic forms; suppositories; injectables; respiratory forms and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance, to ease handling and administration of dose, for convenience of use, or to control bioavailability. While pharmaceutical excipients are commonly described as being inert or inactive, it is appreciated in the art that there is a relationship between the properties of the pharmaceutical excipients and the dosage forms containing them. Pharmaceutical excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995). The concentration of a therapeutically active agent in a formulation can vary widely, from about 0.1 to 99.9 wt. %, depending on the nature of the formulation.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colorants, flavorants, sweetening agents, polymers, waxes or other solubility-modulating materials.

Dosage forms for parenteral administration will generally comprise fluids, particularly intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, The Science and Practice of Pharmacy [full citation previously provided], and include:
alcohol, e.g., 5% alcohol (e.g., in dextrose and water (“D/W”) or D/W in normal saline solution (“NSS”), including in 5% dextrose and water (“D5/W”), or D5/W in NSS);
synthetic amino acid such as Aminosyn, FreAmine, Travasol, e.g., 3.5 or 7; 8.5; 3.5, 5.5 or 8.5% respectively;
ammonium chloride e.g., 2.14%;
dextran 40, in NSS e.g., 10% or in D5/W e.g., 10%;
dextran 70, in NSS e.g., 6% or in D5/W e.g., 6%;
dextrose (glucose, D5/W) e.g., 2.5-50%;
dextrose and sodium chloride e.g., 5-20% dextrose and 0.22-0.9% NaCl;
lactated Ringer's (Hartmann's) e.g., NaCl 0.6%, KCl 0.03%, CaCl₂ 0.02%;
lactate 0.3%;
mannitol e.g., 5%, optionally in combination with dextrose e.g., 10% or NaCl e.g., 15 or 20%;
multiple electrolyte solutions with varying combinations of electrolytes, dextrose, fructose, invert sugar Ringer's e.g., NaCl 0.86%, KCl 0.03%, CaCl₂ 0.033%;
sodium bicarbonate e.g., 5%;
sodium chloride e.g., 0.45, 0.9, 3, or 5%;
sodium lactate e.g., 1/6 M; and
sterile water for injection
   The pH of such IV fluids may vary, and will typically be from 3.5 to 8 as known in the art.

The administration of the anti-mitotic agents of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the anti-mitotic agents may be directly applied as a solution or spray.

To employ the compounds of the invention in a method of screening for compounds that bind to KSP kinesin, the KSP is bound to a support, and a compound of the invention (which is an anti-mitotic agent) is added to the assay. Alternatively, the compound of the invention is bound to the support and KSP is added. Classes of compounds among which novel binding agents may be sought include specific antibodies, non-natural binding agents identified in screens of chemical libraries, peptide analogs, etc. Of particular interest are screening assays for candidate agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the anti-mitotic agent to KSP may be done in a number of ways. In a preferred embodiment, the anti-mitotic agent (the compound of the invention) is labeled, for example, with a fluorescent or radioactive moiety and binding determined directly. For example, this may be done by attaching all or a portion of KSP to a solid support, adding a labeled anti-mitotic agent (for example a compound of the invention in which at least one atom has been replaced by a detectable isotope), washing off excess reagent, and determining whether the amount of the label is that present on the solid support. Various blocking and washing steps may be utilized as is known in the art.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the kinesin proteins may be labeled at tyrosine positions using ¹²⁵I, or with fluorophores. Alternatively, more than one component may be labeled with different labels; using 125I for the proteins, for example, and a fluorophor for the anti-mitotic agents.

The compounds of the invention may also be used as competitors to screen for additional drug candidates. “Candidate bioactive agent” or “drug candidate” or grammatical equivalents as used herein describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. Screens of this sort may be performed either in the presence or absence of microtubules. In the case where protein binding or activity is screened, preferred embodiments exclude molecules already known to bind to that particular protein, for example, polymer structures such as microtubules, and energy sources such as ATP. Preferred embodiments of assays herein include candidate agents which do not bind the cellular proliferation protein in its endogenous native state termed herein as “exogenous” agents. In another preferred embodiment, exogenous agents further exclude antibodies to KSP.

Candidate agents can encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding and lipophilic binding, and typically include at least an amine, carbonyl, hydroxyl, ether, or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

Competitive screening assays may be done by combining KSP and a drug candidate in a first sample. A second sample comprises a anti-mitotic agent, KSP and a drug candidate. This may be performed in either the presence or absence of microtubules. The binding of the drug candidate is determined for both samples, and a change, or difference in binding between the two samples indicates the presence of an agent capable of binding to KSP and potentially modulating its activity. That is, if the binding of the drug candidate is different in the second sample relative to the first sample, the drug candidate is capable of binding to KSP.

In a preferred embodiment, the binding of the candidate agent is determined through the use of competitive binding assays. In this embodiment, the competitor is a binding moiety known to bind to KSP, such as an antibody, peptide, binding partner, ligand, etc. Under certain circumstances, there may be competitive binding as between the candidate agent and the binding moiety, with the binding moiety displacing the candidate agent.

In one embodiment, the candidate agent is labeled. Either the candidate agent, or the competitor, or both, is added first to KSP for a time sufficient to allow binding, if present. Incubations may be performed at any temperature which facilitates optimal activity, typically between 4 and 40° C.

Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the candidate agent. Displacement of the competitor is an indication the candidate agent is binding to KSP and thus is capable of binding to, and potentially modulating, the activity of KSP. In this embodiment, either component can be labeled. Thus, for example, if the competitor is labeled, the presence of label in the wash solution indicates displacement by the agent. Alternatively, if the candidate agent is labeled, the presence of the label on the support indicates displacement.

In an alternative embodiment, the candidate agent is added first, with incubation and washing, followed by the competitor. The absence of binding by the competitor may indicate the candidate agent is bound to KSP with a higher affinity. Thus, if the candidate agent is labeled, the presence of the label on the support, coupled with a lack of competitor binding, may indicate the candidate agent is capable of binding to KSP.

It may be of value to identify the binding site of KSP. This can be done in a variety of ways. In one embodiment, once KSP has been identified as binding to the anti-mitotic agent, KSP is fragmented or modified and the assays repeated to identify the necessary components for binding.

Modulation is tested by screening for candidate agents capable of modulating the activity of KSP comprising the steps of combining a candidate agent with KSP, as above, and determining an alteration in the biological activity of KSP. Thus, in this embodiment, the candidate agent should both bind to KSP (although this may not be necessary), and alter its biological or biochemical activity as defined herein. The methods include both in vitro screening methods and in vivo screening of cells for alterations in cell cycle distribution, cell viability, or for the presence, morpohology, activity, distribution, or amount of mitotic spindles, as are generally outlined above.

Alternatively, differential screening may be used to identify drug candidates that bind to the native KSP, but cannot bind to modified KSP.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of particular and preferred groups described herein above.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

General Methods

The compounds of this invention may be prepared according to the general process outlined in Schemes 1-4 and described in Examples 1-12 below.

The following abbreviations and terms have the indicated meanings throughout: Ac represents acetyl, BNB represents 4-bromomethyl-3-nitrobenzoic acid, Boc represents t-butyloxy carbonyl, br represents broad, Bu represents butyl, c-represents cyclo, CBZ represents carbobenzoxy represents benzyloxycarbonyl, d represents doublet, DBU represents diazabicyclo[5.4.0]undec-7-ene, DCM represents dichloromethane represents methylene chloride represents CH₂Cl₂, DCE represents dichloroethylene, DEAD represents diethyl azodicarboxylate, DIC represents diisopropylcarbodiimide, DIEA represents N,N-diisopropylethyl amine, DMAP represents 4-N,N-dimethylaminopyridine, DMF represents N,N-dimethylformamide, DMSO represents dimethyl sulfoxide, DVB represents 1,4-divinylbenzene, EEDQ represents 2-ethoxy-1-ethoxycarbonyl-1,2-dihydroquinoline, ESMS represents electrospray mass spectrometry, Et represents ethyl, Fmoc represents 9-fluorenylmethoxycarbonyl, GC represents gas chromatography, HATU represents O-(7-Azabenzotriazol-1-yl)-1,1,3,3-tetramethyluronium, hexafluorophosphate, HMDS represents hexamethyldisilazane, HOAc represents acetic acid, HOBt represents hydroxybenzotriazole, HPLC represents high pressure liquid chromatography, m represents multiplet, Me represents methyl, Ms represents methanesulfonyl, MHz represents megahertz, MTBE represents methyl t-butyl ether, NMO represents N-methylmorpholine oxide, NMR represents nuclear magnetic resonance, PEG represents polyethylene glycol, Ph represents phenyl, PhOH represents phenol, PfP represents pentafluorophenol, PPTS represents pyridinium p-toluenesulfonate, Py represents pyridine, PyBroP represents bromo-tris-pyrrolidino-phosphonium hexafluorophosphate, rt or RT represent room temperature, sat'd represents saturated, s represents singlet, s-represents secondary, t represents triplet, t-represents tertiary, TBDMS represents t-butyidimethylsilyl, TES represents triethylsilyl, TFA represents trifluoroacetic acid, THF represents tetrahydrofuran, TMOF represents trimethyl orthoformate, TMS represents trimethylsilyl, tosyl represents p-toluenesulfonyl, and Trt represents triphenylmethyl.

Compounds of formula (I) can be readily prepared via a cross-coupling reaction between a suitable aryl halide and a suitable aryl organometallic agent under standard conditions (Scheme 1). The coupling of suitable aryl halides, such as when X is iodine, bromine, or chlorine, and suitable aryl organometallic agents, such as when M is (functionalized) boron, magnesium, or tin, is discussed in depth in the literature. Aryl halides and, as an example, aryl boronic acids/esters are either commercially available, reported in the literature, or can be prepared following literature procedures by those skilled in the art.

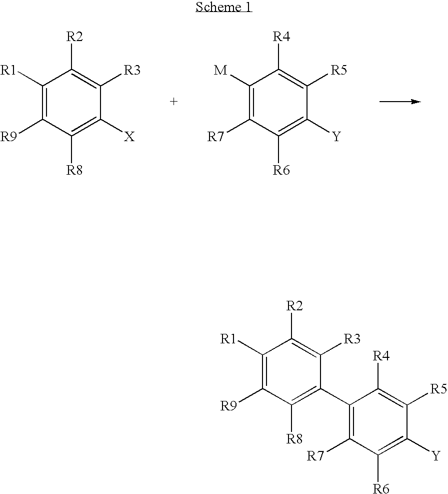

Suzuki cross-coupling of an aryl halide (such as a functionalized bromoaniline) with a functionalized aryl boronic acid using a palladium catalyst (typically tetrakis(triphenylphosphine)palladium(0)) in the presence of a base (such as potassium carbonate solution) and a suitable solvent (such as N,N-dimethylformamide) at elevated temperatures (for example, 100° C.) affords a functionalized biarylaniline (Scheme 2). This reaction can also be performed when the coupling partners are switched, i.e. the aniline moiety is the boronic acid and the aryl boronic acid is the aryl halide.

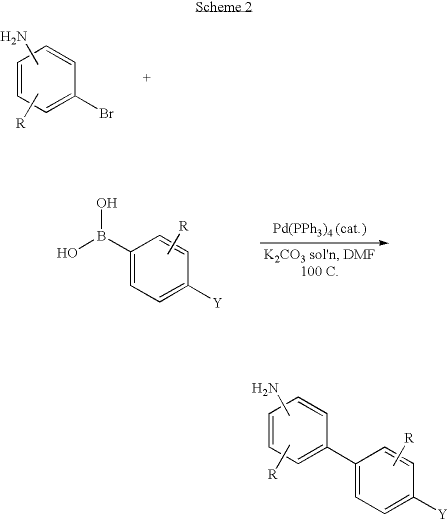

Such biarylanilines can then undergo sulfamoylation under standard conditions using functionalized sulfamoyl chlorides. For example, sulfamoylation of a functionalized biarylaniline with a sulfamoyl chloride [See Lee, C.-H.; Kohn, H. J. Org. Chem. 1990, 55, 6098-6104; Cohen, E.; Klarberg, B. J. Am. Chem. Soc. 1962, 84,1994-2002.] in the presence of a suitable base (such as pyridine) and optionally a solvent (such as acetonitrile) at room temperature or elevated temperature provides a functionalized biarylsulfamide (Scheme 3).

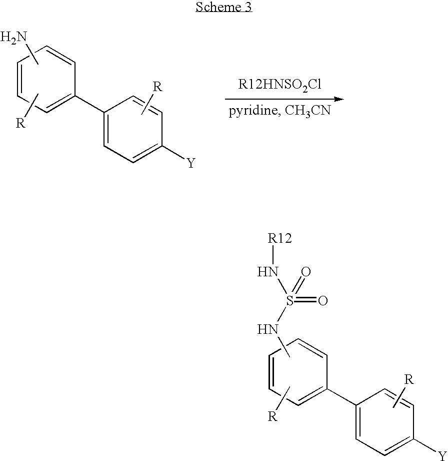

Alternatively, functionalized biarylanilines can undergo sulfamoylation utilizing commercially available sulfamide. For example, heating of a functionalized biarylaniline with excess sulfamide in a suitable solvent (such as 1,4-dioxane, THF, or pyridine) at elevated temperatures (such as at reflux) or by irradiation in the microwave provides the functionalized biarylsulfamide (Scheme 4). 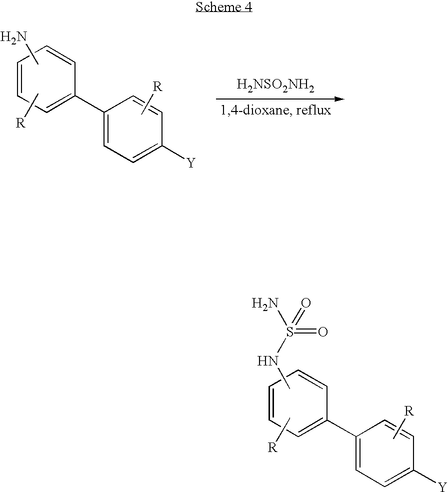
Synthesis of Compounds

### PREPARATION 1

### 4′-(trifluoromethyl)-4-biphenylamine

A solution of 4-bromoaniline (29 mmol), 4-trifluoromethylphenyl boronic acid (35 mmol), and tetrakis(triphenylphosphine)palladium(0) (1.4 mmol) in 2M aqueous potassium carbonate solution (50 mL) and N,N-dimethylformamide (50 mL) was heated at 100° C. for 17 h. The reaction mixture was cooled, poured into half-saturated aqueous sodium bicarbonate solution (400 mL), and extracted with (3×400 mL) diethyl ether. The combined organic layers were dried over sodium sulfate and concentrated in vacuo. Purification of the residue by flash chromatography (10-30% ethyl acetate/hexanes) provided the title product as a white powder (70%). ESMS [M+H]⁺: 238.2.

### PREPARATION 2

### [3-fluoro-4′-(trifluoromethyl)-4-biphenylyl]amine

Following the procedure described in Preparation 1 with 4-bromo-2-fluoroaniline provided the title compound. ESMS [M+H]⁺: 256.2.

### PREPARATION 3

### [4-(2,2,4,4-tetrafluoro-4H-1,3-benzodioxin-6-yl)phenyl]amine

Following the procedure described in Preparation 1 with 4-(4,4,5,5-tetramethyl-1,3,2-dioxaborolan-2-yl)aniline and 6-bromo-2,2,4,4-tetrafluoro-4H-1,3-benzodioxin and purification by Gilson reverse phase HPLC provided the title compound. ESMS [M+H]⁺: 300.2.

### PREPARATION 4

### 4′-(trifluoromethyl)-3-biphenylamine

Following the procedure described in Preparation 1 with 3-bromoaniline provided the title compound. ESMS [M+H]⁺: 238.2.

### PREPARATION 5

### 3′-fluoro-4′-(trifluoromethyl)-4-biphenylamine

Following the procedure described in Preparation 1 with 4-bromo-2-fluoro-1-(trifluoromethyl)benzene and 4-(4,4,5,5-tetramethyl-1,3,2-dioxaborolan-2-yl)aniline provided the title compound. ESMS [M+H]⁺: 256.2.

### PREPARATION 6

### 4′-[(trifluoromethyl)thio]-4-biphenylamine

Following the procedure described in Preparation 1 with 4-bromophenyl trifluoromethyl sulfide and 4-(4,4,5,5-tetramethyl-1,3,2-dioxaborolan-2-yl)aniline provided the title compound. ESMS [M+H]⁺: 270.2.

### PREPARATION 7

### 4′-[(trifluoromethyl)sulfonyl]-4-biphenylamine

Following the procedure described in Preparation 1 with 4-chlorophenyl trifluoromethyl sulfone and 4-(4,4,5,5-tetramethyl-1,3,2-dioxaborolan-2-yl)aniline provided the title compound. ESMS [M+H]⁺: 302.2.

### PREPARATION 8

### 4-amino-4′-(trifluoromethyl)-3-biphenylcarbonitrile

Following the procedure described in Preparation 1 with 2-amino-5-bromobenzonitrile provided the title compound. ESMS [M+H]⁺: 263.2.

### PREPARATION 9

### 3-(methoxy)-4-nitro-4′-(trifluoromethyl)biphenyl

Following the procedure described in Preparation 1 with 5-chloro-2-nitroanisole provided the title compound. ESMS [M+H]⁺: 298.2.

### PREPARATION 10

### [3-nitro-4′-(trifluoromethyl)-4-biphenylyl]amine

Following the procedure described in Preparation 1 with 4-bromo-2-nitroaniline provided the title compound. ESMS [M+H]⁺: 283.2.

### PREPARATION 11

### N-methyl-4-nitro-4′-(trifluoromethyl)-3-biphenylamine

Following the procedure described in Preparation 1 with 5-chloro-N-methyl-2-nitroaniline provided the title compound. ESMS [M+H]⁺: 297.2.

### PREPARATION 12

### 3-fluoro-4-nitro-4′-(trifluoromethyl)biphenyl

Following the procedure described in Preparation 1 with 4-chloro-2-fluoronitrobenzene in 1,4-dioxane provided the title compound. ESMS [M+H]⁺: 286.0.

### PREPARATION 13

### 2-methyl-4′-(trifluoromethyl)-4-biphenylamine

Following the procedure described in Preparation 1 with 4-bromo-3-methylaniline provided the title compound.

### PREPARATION 14

### 3-methyl-4′-(trifluoromethyl)-4-biphenylamine

Following the procedure described in Preparation 1 with 4-bromo-2-methylaniline provided the title compound.

### PREPARATION 15

### 2-fluoro-4′-(trifluoromethyl)-4-biphenylamine

Following the procedure described in Preparation 1 with 4-bromo-3-fluoroaniline provided the title compound.

### PREPARATION 16

### 2,5-difluoro-4′-(trifluoromethyl)-4-biphenylamine

Following the procedure described in Preparation 1 with 4-bromo-2,5-difluoroaniline provided the title compound.

### PREPARATION 17

### 2,6-difluoro-4′-(trifluoromethyl)-4-biphenylamine

Following the procedure described in Preparation 1 with 4-bromo-3,5-difluoroaniline provided the title compound.

### PREPARATION 18

### 2-chloro-4′-(trifluoromethyl)-4-biphenylamine

Following the procedure described in Preparation 1 with 4-bromo-3-chloroaniline provided the title compound.

### PREPARATION 19

### 3-chloro-4′-(trifluoromethyl)-4-biphenylamine

Following the procedure described in Preparation 1 with 4-bromo-2-chloroaniline provided the title compound.

### PREPARATION 20

### 2-(methyloxy)-4′-(trifluoromethyl)-4-biphenylamine

Following the procedure described in Preparation 1 with 4-bromo-3-(methyloxy)aniline provided the title compound.

### PREPARATION 21

### 2-(hydroxvmethyl)-4′-(trifluoromethyl)-4-biphenylamine

Following the procedure described in Preparation 1 with 4-bromo-3-(hydroxylmethyl)aniline provided the title compound.

### PREPARATION 22

### 3-(hydroxvmethyl)-4′-(trifluoromethyl)-4-biphenylamine

Following the procedure described in Preparation 1 with 4-bromo-2-(hydroxymethyl)aniline provided the title compound.

### PREPARATION 23

### 3-bromo-5-fluoro-4′-(trifluoromethyl)-4-biphenylamine

Following the procedure described in Preparation 1 with 2,4-dibromo-6-fluoroaniline provided the title compound.

### PREPARATION 24

### 3,5-difluoro-4′-(trifluoromethyl)-4-biphenylamine

Following the procedure described in Preparation 1 with 4-bromo-2,6-difluoroaniline provided the title compound.

### PREPARATION 25

### 3′-nitro-4′-(trifluoromethyl)-4-biphenylamine

Following the procedure in Preparation 1 with 4-aminophenyl boronic acid and 4-bromo-2-nitro-1-(trifluoromethyl)benzene provided the title compound. ESMS [M+H]⁺: 283.2.

### PREPARATION 26

### N-(1-methylethyl)-4-nitro-4′-(trifluoromethyl)-3-biphenylamine

A solution of 3-fluoro-4-nitro-4′-(trifluoromethyl)biphenyl (0.70 mmol), isopropylamine (1.40 mmol) and potassium carbonate (1.40 mmol) in dimethyl sulfoxide (5.0 mL) was irradiated in a Personal Chemistry Emrys Optimizer microwave at 150° C. for 5 min. Ethyl acetate (10 mL) was added and the mixture was washed with water (3×10 mL). The organic layer was concentrated in vacuo to give the title compound as a solid. ESMS [M+H]⁺: 325.2.

### PREPARATION 27

### [4-(2,2,3,3-tetrafluoro-2,3-dihydro-1,4-benzodioxin-6-yl)phenyl]amine

A solution of 5-bromo-2,2,3,3-tetrafluoro-1,4-benzodioxane (0.340 g, 1.20 mmol), [4-(4,4,5,5-tetramethyl-1,3,2-dioxaborolan-2-yl)phenyl]amine (0.289 g, 1.32 mmol), cesium carbonate (0.676 g, 2.08 mmol), and dichloro[1,1′-bis(diphenylphosphino)ferrocene]palladium(II)•dichloromethane adduct (0.015 g, 0.02 mmol) in N,N-dimethylformamide (6 mL) was irradiated in a Personal Chemistry Emrys Optimizer microwave at 110° C. for 10 min. The reaction mixture was diluted with diethyl ether and washed with saturated aqueous sodium bicarbonate solution. The organic layer was evaporated to give 0.275 g of crude product. ESMS [M+H]⁺: 300.0.

### PREPARATION 28

### N-[3-(methyloxy)-4′-(trifluoromethyl)-4-biphenylyl]amine

To a solution of 3-(methyloxy)-4-nitro-4′-(trifluoromethyl)biphenyl (1.96 mmol) in acetic acid (10 mL) was added zinc dust (13.7 mmol). The reaction mixure was stirred at room temperature for 2 h and then filtered through Celite and washed with acetic acid (5 mL) and ethanol (5 mL). The filtrate was concentrated in vacuo and the residue was taken up into ethyl acetate (15 mL) and washed with saturated aqueous sodium carbonate solution (10 mL). The organic layer was dried over magnesium sulfate and concentrated in vacuo to give the title compound as an off-white solid. ESMS [M+H]⁺: 268.0.

### PREPARATION 29

### [4-amino-4′-(trifluoromethyl)-3-biphenylyl]methylamine

Following the procedure described in Preparation 28 with N-methyl-4-nitro-4′-(trifluoromethyl)-3-biphenylamine provided the title compound. ESMS [M+H]⁺: 267.0.

### PREPARATION 30

### [4-amino-4′-(trifluoromethyl)-3-biphenylyl](1-methylethyl)amine

Following the procedure described in Preparation 28 with N-(1-methylethyl)-4-nitro-4′-(trifluoromethyl)-3-biphenylamine provided the title compound. ESMS [M+H]⁺: 295.0.

### PREPARATION 31

### 4-amino-4′-(trifluoromethyl)-3-biphenylol

To a solution of [3-(methyloxy)-4′-(trifluoromethyl)-4-biphenylyl]amine (0.68 mmol) in 1-methyl-2-pyrrolidinone (0.34 mL) was added potassium carbonate (0.034 mmol) and thiophenol (0.68 mmol). The reaction mixture was irradiated in a Personal Chemistry Emrys Optimizer microwave at 220° C. for 15 min. The reaction mixture was diluted with ethyl acetate (10 mL) and concentrated in vacuo. Purification of the residue by silica gel chromatography (Isco-Combiflash, 20-65% ethyl acetate/hexanes) afforded the title compound as a brown solid (82%). ESMS [M+H]⁺: 254.2.

### PREPARATION 32

### 2-[3-fluoro-4-(trifluoromethyl)phenyl]-4,4,5,5-tetramethyl-1,3,2-dioxaborolane

A solution of 4-bromo-2-fluoro-1-(trifluoromethyl)benzene (20.57 mmol), 4,4,4′,4′,5,5,5′,5′-octamethyl-2,2′-bi-1,3,2-dioxaborolane (22.62 mmol), potassium acetate (61.70 mmol), and dichlorobis(triphenylphosphine)palladium(II) (0.822 mmol) in dioxane (200 mL) was heated at 100° C. for 4.5 h. The reaction mixture was cooled, dissolved into ethyl acetate (500 mL), and washed with water (3×200 mL). The organic layer was dried over sodium sulfate and concentrated in vacuo to provide the crude title product.

### PREPARATION 33

### 3′-fluoro-2-methyl-4′-(trifluoromethyl)-4-biphenylamine

A solution of 4-bromo-3-methylaniline (0.34 mmol), 2-[3-fluoro-4-(trifluoromethyl)phenyl]-4,4,5,5-tetramethyl-1,3,2-dioxaborolane (0.52 mmol), and tetrakis(triphenylphosphine)palladium(0) (0.017mmol) in 2M aqueous potassium carbonate solution (1 mL) and N,N-dimethylformamide (1 mL) was heated at 100° C. for 17 h. The reaction mixture was cooled, poured into half-saturated aqueous sodium bicarbonate solution (2 mL), and extracted with (3×5 mL) diethyl ether. The combined organic layers were dried over sodium sulfate and concentrated in vacuo to afford the crude title product.

### PREPARATION 34

### 2,3′-difluoro-4′-(trifluoromethyl)-4-biphenylamine

Following the procedure described in Preparation 33 with 4-bromo-3-fluoroaniline provided the title compound.

### PREPARATION 35

### 2,3′,5-trifluoro-4′-(trifluoromethyl)-4-biphenylamine

Following the procedure described in Preparation 33 with 4-bromo-2,5-difluoroaniline provided the title compound.

### PREPARATION 36

### 2,3′,6-trifluoro-4′-(trifluoromethyl)-4-biphenylamine

Following the procedure described in Preparation 33 with 4-bromo-3,5-difluoroaniline provided the title compound.

### PREPARATION 37

### 2-chloro-3′-fluoro-4′-(trifluoromethyl)-4-biphenylamine

Following the procedure described in Preparation 33 with 4-bromo-3-chloroaniline provided the title compound.

### PREPARATION 38

### 3-chloro-3′-fluoro-4′-(trifluoromethyl)-4-biphenylamine

Following the procedure described in Preparation 33 with 4-bromo-2-chloroaniline provided the title compound.

### PREPARATION 39

### 3,3′,5-trifluoro-4′-(trifluoromethyl)-4-biphenylamine

Following the procedure described in Preparation 33 with 4-bromo-2,6-difluoroaniline provided the title compound.

### PREPARATION 40

### 3′-fluoro-2-(methyloxy)-4′-(trifluoromethyl)-4-biphenylamine

Following the procedure described in Preparation 33 with 4-bromo-3-(methyloxy)aniline provided the title compound.

### PREPARATION 41

### 3′-fluoro-3-methyl-4′-(trifluoromethyl)-4-biphenylamine

Following the procedure described in Preparation 33 with 4-bromo-2-methylaniline provided the title compound.

### PREPARATION 42

### 3-ethyl-3′-fluoro-4′-(trifluoromethyl)-4-biphenylamine

Following the procedure described in Preparation 33 with 4-bromo-2-ethylaniline provided the title compound.

### PREPARATION 43

### 3′-fluoro-3-(hydroxymethyl)-4′-(trifluoromethyl)-4-biphenylamine

Following the procedure described in Preparation 33 with 4-bromo-2-(hydroxymethyl)aniline provided the title compound.

### PREPARATION 44

### 3,3′-difluoro-4′-(trifluoromethyl)-4-biphenylamine

To a solution of 2-fluoro-4-iodoaniline (3.50 g, 14.8 mMol), bis(pinacolato)diboron (4.88 g, 19.2 mMol), and potassium acetate (4.35 g, 44.3 mMol) in N,N-dimethylformamide (80 mL) was added dichloro[1,1′-bis(diphenylphosphino)ferrocene]palladium(ll)•dichloromethane adduct (0.33 g, 0.45 mMol). The reaction mixture was stirred at 80° C. for 4 h, at which point 4-bromo-2-fluoro-1-(trifluoromethyl)benzene (3.6 g, 14.8 mMol), potassium carbonate (10.2 g, 73.8 mMol), water (20 mL), and additional dichloro[1,1′-bis(diphenylphosphino)ferrocene]palladium(II)•dichloromethane adduct (0.33 g, 0.45 mMol) were added. The reaction mixture was heated at 100° C. for 18 h and then cooled to room temperature. The reaction mixture was concentrated in vacuo and the residue was dissolved in ethyl acetate, filtered to remove insolubles, washed with brine, dried over sodium sulfate, and concentrated in vacuo. Purification of the residue by silica gel chromatography (20% ethyl acetate/hexanes), trituration with hexanes, filtration, and concentration in vacuo provided the title compound (2.30 g, 57%) as a white solid. ESMS [M+H]⁺: 274.2.

### PREPARATION 45

### 3-fluoro-3′-nitro-4′-(trifluoromethyl)-4-biphenylamine

To a solution of 1,4-dibromo-2-nitrobenzene (5.0 g, 17.8 mMol) in 1-methyl-2-pyrrolidinone (25 mL) was added copper(I) iodide (0.85 g, 4.5 mMol) and methyl 2-(fluorosulfonyl)difluoroacetate (4.6 mL, 36.1 mMol). The reaction mixture was heated at 80° C. for 24 h. After cooling to room temperature, the reaction mixture was diluted with water and the organics were extracted with ethyl acetate, filtered to remove insolubles, washed with brine, dried over magnesium sulfate, and concentrated in vacuo. Purification of the residue by silica gel chromatography (5% ethyl acetate/hexanes) gave the intermediate 4-bromo-2-nitro-1-(trifluoromethyl)benzene (4.10 g, 85%) as a yellow oil. ¹H NMR (400 MHz, CDCl₃) δ 8.06 (d, 1H, J=1.5 Hz), 7.87-7.92 (m, 1H), 7.73 (d, 1H, J=8.3 Hz).

Following the procedure described in Preparation 44 with 4-bromo-2-nitro-1-(trifluoromethyl)benzene provided the title compound as a yellow solid (63%). ESMS [M+H]⁺: 301.2.

### PREPARATION 46

### 3-fluoro-5-nitro-4′-(trifluoromethyl)-4-biphenylamine

To a ice-cooled solution of 3-fluoro-4′-(trifluoromethyl)-4-biphenylamine (1.17 mmol) in concentrated sulfuric acid (2.0 mL) was added 90% nitric acid (1.41 mmol). The reaction mixture warmed to room temperature and stirred for 2 h. The reaction mixture was then poured into ice water (60 mL) and the resultant orange solid was filtered and dried in vacuo to afford the crude title compound.

### PREPARATION 47

### 4-(2,2-difluoro-1,3-benzodioxol-5-yl)aniline

A solution of 5-bromo-2,2-difluoro-1,3-benzodioxole (2.65 mmol), 4-(4,4,5,5-tetramethyl-1,3,2-dioxaborolan-2-yl)aniline (3.97 mmol), tetrakis(triphenylphosphine)palladium(0) (0.08 mmol), and cesium carbonate (7.94 mmol) in N,N-dimethylformamide (8.0 mL) and water (2.0 mL) was heated at 100° C. for 18 h. The reaction mixture was cooled, poured into brine (60 mL), and extracted with ethyl acetate (3×50 mL). The combined organic layers were dried over magnesium sulfate and decolorizing charcoal, filtered through Celite, and concentrated in vacuo. Purification of the residue by Gilson reverse phase HPLC and neutralization of the collected fractions afforded the title product as a white solid (50%). ESMS [M+H]⁺: 250.2.

### PREPARATION 48

### 4-(1,3-benzodioxol-5-yl)aniline

A solution of 3,4-(methylenedioxy)phenylboronic acid (5.14mmol), 4-iodoaniline (3.42 mmol), dichloro[1,1′-bis(diphenylphosphino)ferrocene]palladium(II)•dichloromethane adduct (0.10 mmol), and cesium carbonate (17.1 mmol) in N,N-dimethylformamide (10 mL) and water (3 mL) was heated at 100° C. for 20 h. The reaction mixture was cooled, poured into brine (50 mL), and extracted with (3×50 mL) ethyl acetate. The combined organic layers were dried over magnesium sulfate and decolorizing charcoal, filtered through Celite, and concentrated in vacuo. Purification of the residue by Gilson reverse phase HPLC and neutralization of the collected fractions afforded the title product as an off white solid (60%). ESMS [M+H]⁺: 214.1.

### PREPARATION 49

### 4-(1,3-benzodioxol-5-yl)-2-fluoroaniline

Following the procedure described in Preparation 48 with 2-fluoro-4-iodoaniline provided the title compound. ESMS [M+H]⁺: 232.2.

### PREPARATION 50

### 4′-amino-3′-fluoro-4-(trifluoromethyl)-2-biphenylcarbonitrile

A solution of 4-BOC-amino-3-fluorophenylboronic acid (2.76 mmol), 2-chloro-5-(trifluoromethyl)benzonitrile (1.84 mmol), dichloro[1,1′-bis(diphenylphosphino)ferrocene]palladium(II)•dichloromethane adduct (0.06 mmol), and cesium carbonate (9.2 mmol) in N,N-dimethylformamide (8 mL) and water (2 mL) was heated at 100° C. for 20 h. The reaction mixture was cooled, poured into brine (50 mL), and extracted with (3×50 mL) ethyl acetate. The combined organic layers were dried over magnesium sulfate and decolorizing charcoal, filtered through Celite, and concentrated in vacuo. Purification of the residue by Gilson reverse phase HPLC afforded the protected intermediate as a pale orange solid. This solid was treated with a solution of dichloromethane (10 mL) and trifluoroacetic acid (4 mL) and stirred for 2 h. Concentration in vacuo and subsequent neutralization afforded the title product as a pale tan solid (36%). ESMS [M+H]⁺: 281.4.

### PREPARATION 51

### 4′-[(trifluoromethyl)sulfinyl]-4-biphenylamine

To a stirred solution of 4′-[(trifluoromethyl)thio]-4-biphenylamine (3.71 mmol) in dry dichloromethane (40 mL) at 0° C. under nitrogen was added portionwise m-chloroperoxybenzoic acid (4.82 mmol). The reaction mixture was stirred for 6 h at 0° C. and then at room temperature for 1 h. The crude solution was filtered, evaporated, and purified by flash chromatography (20-30% ethyl acetate/hexanes) to provide the title product as an orange solid (85%). ESMS [M+H]⁺: 286.2.

### PREPARATION 52

### 4-amino-4′-(trifluoromethyl)-2-biphenylcarboxamide

To a solution of 2-chloro-5-nitrobenzamide (2.73 mmol) in acetic acid (20 mL) was added zinc dust (27.3 mmol). The reaction mixure was stirred at room temperature for 2 hours and then filtered through Celite and washed with acetic acid (10 mL) and ethanol (10 mL). The filtrate was concentrated in vacuo and the residue was taken up into ethyl acetate (30 mL) and washed with saturated aqueous sodium carbonate solution (20 mL). The organic layer was dried over magnesium sulfate and concentrated in vacuo to give 5-amino-2-chlorobenzamide as a solid.

A solution of 5-amino-2-chlorobenzamide (1.79 mmol), 4-trifluoromethylphenyl boronic acid (2.68 mmol), palladium(II) acetate (8.95 umol), potassium phosphate (3.58 mmol), 2-dicyclohexylphosphino-2′-(N,N-dimethylamino)biphenyl (0.013 mmol) in 1,4-dioxane (5.4 mL) was heated at 100° C. for 18 h. The reaction mixture was cooled, diluted with saturated aqueous sodium bicarbonate solution (5 mL), and extracted with (3×5 mL) ethyl acetate. The combined organic layers were dried over magnesium sulfate and concentrated in vacuo. Purification of the residue by flash chromatography (10-90% ethyl acetate/hexanes) provided the title product as a white powder (55%). ESMS [M+H]⁺: 281.0.

### EXAMPLE 1

### Ex 1a N-[4′-(trifluoromethyl)-4-biphenylyl]sulfamide

To an ice-cooled solution of chlorosulfonylisocyanate (0.47 mmol) in acetonitrile (1.0 mL) was added water (0.47 mmol). The reaction mixture was stirred at 0° C. for 1 min and then allowed to warm to room temperature slowly. After 3 h, the reaction mixture was cooled to 0° C., at which point a solution of 4′-(trifluoromethyl)-4-biphenylamine (0.43 mmol), pyridine (0.86 mmol), and acetonitrile (1.0 mL) was added dropwise. The reaction mixture was stirred at 0° C. for 1 min and then allowed to warm to room temperature slowly. After 15 h, the reaction mixture was diluted with water (60 mL) and brine (10 mL), and extracted with (3×50 mL) ethyl acetate. The combined organic layers were dried over sodium sulfate and then concentrated in vacuo. Purification by silica gel chromatography (Isco Combi-Flash, 20-50% ethyl acetate/hexanes) afforded the title compound as a white solid (57%). ESMS [M+H]⁺: 317.2.

### Ex 1b N-[3-fluoro-4′-(trifluoromethyl)-4-biphenylyl]sulfamide

Following the procedure described in Example 1 a with [3-fluoro-4′-(trifluoromethyl)-4-biphenylyl]amine provided the title product. ESMS [M+H]⁺: 355.0.

### Ex 1c N-[4-(2,2,4,4-tetrafluoro-4H-1,3-benzodioxin-6-yl)phenyl]sulfamide

Following the procedure described in Example 1a with [4-(2,2,4,4-tetrafluoro-4H-1,3-benzodioxin-6-yl)phenyl]amine and purification by Gilson reverse phase HPLC provided the title product. ESMS [M+H]⁺: 379.2.

### Ex 1d N-[4-(2,2-difluoro-1,3-benzodioxol-5-yl)phenyl]sulfamide

Following the procedure described in Example 1a with 4-(2,2-difluoro-1,3-benzodioxol-5-yl)aniline provided the title product. ESMS [M+H]⁺: 329.3.

### Ex 1e N-[4-(1,3-benzodioxol-5-yl)phenyl]sulfamide

Following the procedure described in Example 1a with 4-(1,3-benzodioxol-5-yl)aniline provided the title product. ESMS [M+H]⁺: 293.2.

### Ex 1f N-[4-(1,3-benzodioxol-5-yl)-2-fluorophenyl]sulfamide

Following the procedure described in Example 1a with 4-(1,3-benzodioxol-5-yl)-2-fluoroaniline provided the title product. ESMS [M+H]⁺: 311.4.

### Ex 1g N-[2′-cvano-3-fluoro-4′-(trifluoromethyl)-4-biphenylyl]sulfamide

Following the procedure described in Example 1a with 4′-amino-4-(trifluoromethyl)-2-biphenylcarbonitrile provided the title product. ESMS [M+H]⁺: 360.2.

### Ex 1h N-[4′-(trifluoromethyl)-3-biphenylyl]sulfamide

Following the procedure described in Example 1a with 4′-(trifluoromethyl)-3-biphenylamine provided the title product. ESMS [M+H]⁺: 317.2.

### Ex 1i N-[3′-fluoro-4′-(trifluoromethyl)-4-biphenylyl]sulfamide

Following the procedure described in Example 1a with 3′-fluoro-4′-(trifluoromethyl)-4-biphenylamine provided the title product. ESMS [M+H]⁺: 355.2.

### Ex 1j N-{4′-[(trifluoromethyl)sulfonyl]-4-biphenylyl}sulfamide

Following the procedure described in Example 1a with 4′-[(trifluoromethyl)sulfonyl]-4-biphenylamine provided the title product. ESMS [M+H]⁺: 381.2.

### Ex 1k N-{4′-[(trifluoromethyl)sulfinyl]-4-biphenylyl}sulfamide

Following the procedure described in Example 1a with 4′-[(trifluoromethyl)sulfinyl]-4-biphenylamine provided the title product. ESMS [M+H]⁺: 365.4.

### Ex 1l N-[3-cyano-4′-(trifluoromethyl)-4-biphenylyl]sulfamide

Following the procedure described in Example 1a with 4-amino-4′-(trifluoromethyl)-3-biphenylcarbonitrile provided the title product. ESMS [M+H]⁺: 342.0 [M+Na]⁺: 364.2.

### Ex 1m N-[2-cyano-4′-(trifluoromethyl)-4-biphenylyl]sulfamide

Following the procedure described in Example 1a with 4-amino-4′-(trifluoromethyl)-2-biphenylcarboxamide provided the title product. ESMS [M+H]⁺: 342.0.

### Ex 1n N-[4-(2,2,3,3-tetrafluoro-2,3-dihydro-1,4-benzodioxin-6-yl)phenyl]sulfamide

Following the procedure described in Example 1a with [4-(2,2,3,3-tetrafluoro-2,3-dihydro-1,4-benzodioxin-6-yl)phenyl]amine and purification by Gilson reverse phase HPLC provided the title product. ESMS [M+H]⁺: 379.2.

### Ex 1o N-[3-[(1-methylethyl)amino]-4′-(trifluoromethyl)-4-biphenylyl]sulfamide

Following the procedure described in Example 1a with [4-amino-4′-(trifluoromethyl)-3-biphenylyl](1-methylethyl)amine provided the title compound. ESMS [M+H]⁺: 374.0.

### Ex 1p N-[2-methyl-4′-(trifluoromethyl)-4-biphenylyl]sulfamide

Following the procedure described in Example 1a with 2-methyl-4′-(trifluoromethyl)-4-biphenylamine and purification by Gilson reverse phase HPLC provided the title product. ESMS [M+H]⁺: 331.2.

### Ex 1q N-[3-methyl-4′-(trifluoromethyl)-4-biphenylyl]sulfamide

Following the procedure described in Example 1a with 3-methyl-4′-(trifluoromethyl)-4-biphenylamine and purification by Gilson reverse phase HPLC provided the title product. ESMS [M+H]⁺: 331.2.

### Ex 1r N-[2-fluoro-4′-(trifluoromethyl)-4-biphenylyl]sulfamide

Following the procedure described in Example 1a with 2-fluoro-4′-(trifluoromethyl)-4-biphenylamine and purification by Gilson reverse phase HPLC provided the title product. ESMS [M+H]⁺: 335.4.

### Ex 1s N-[2,5-difluoro-4′-(trifluoromethyl)-4-biphenylyl]sulfamide

Following the procedure described in Example 1a with 2,5-difluoro-4′-(trifluoromethyl)-4-biphenylamine and purification by Gilson reverse phase HPLC provided the title product. ESMS [M+H]⁺: 353.2.

### Ex 1t N-[2,6-difluoro-4′-(trifluoromethyl)-4-biphenylyl]sulfamide

Following the procedure described in Example 1a with 2,6-difluoro-4′-(trifluoromethyl)-4-biphenylamine and purification by Gilson reverse phase HPLC provided the title product. ESMS [M+H]⁺: 353.2.

### Ex 1u N-[2-chloro-4′-(trifluoromethyl)-4-biphenylyl]sulfamide

Following the procedure described in Example 1a with 2-chloro-4′-(trifluoromethyl)-4-biphenylamine and purification by Gilson reverse phase HPLC provided the title product. ESMS [M+H]⁺: 351.2.

### Ex 1v N-[3-chloro-4′-(trifluoromethyl)-4-biphenylyl]sulfamide

Following the procedure described in Example 1a with 3-chloro-4′-(trifluoromethyl)-4-biphenylamine and purification by Gilson reverse phase HPLC provided the title product. ESMS [M+H]⁺: 351.4.

### Ex 1w N-[2-(methyloxy)-4′-(trifluoromethyl)-4-biphenylyl]sulfamide

Following the procedure described in Example 1a with 2-(methyloxy)-4′-(trifluoromethyl)-4-biphenylamine and purification by Gilson reverse phase HPLC provided the title product. ESMS [M+H]⁺: 347.2.

### Ex 1x N-[2-(hydroxymethyl)-4′-(trifluoromethyl)-4-biphenylyl]sulfamide

Following the procedure described in Example 1a with 2-(hydroxymethyl)-4′-(trifluoromethyl)-4-biphenylamine and purification by Gilson reverse phase HPLC provided the title product. ESMS [M+H]⁺: 347.2.

### Ex 1y N-[3-(hydroxymethyl)-4′-(trifluoromethyl)-4-biphenylyl]sulfamide

Following the procedure described in Example 1a with 3-(hydroxymethyl)-4′-(trifluoromethyl)-4-biphenylamine and purification by Gilson reverse phase HPLC provided the title product. ESMS [M+H]⁺: 347.2.

### Ex 1z N-[3-bromo-5-fluoro-4′-(trifluoromethyl)-4-biphenylyl]sulfamide

Following the procedure described in Example 1a with 3-bromo-5-fluoro-4′-(trifluoromethyl)-4-biphenylamine and purification by Gilson reverse phase HPLC provided the title product. ESMS [M+H]⁺: 414.4.

### Ex 1aa N-[3′-fluoro-2-methyl-4′-(trifluoromethyl)-4-biphenylyl]sulfamide

Following the procedure described in Example 1a with 3′-fluoro-2-methyl-4′-(trifluoromethyl)-4-biphenylamine and purification by Gilson reverse phase HPLC provided the title product. ESMS [M+H]⁺: 349.2.

### Ex 1bb N-[2,3′-difluoro-4′-(trifluoromethyl)-4-biphenylyl]sulfamide

Following the procedure described in Example 1a with 2,3′-difluoro-4′-(trifluoromethyl)-4-biphenylamine and purification by Gilson reverse phase HPLC provided the title product. ESMS [M+H]⁺: 353.2.

### Ex 1cc N-[2,3′,5-trifluoro-4′-(trifluoromethyl)-4-biphenylyl]sulfamide

Following the procedure described in Example 1a with 2,3′,5-trifluoro-4′-(trifluoromethyl)-4-biphenylamine and purification by Gilson reverse phase HPLC provided the title product. ESMS [M+H]⁺: 371.2.

### Ex 1dd N-[2,3′,6-trifluoro-4′-(trifluoromethyl)-4-biphenylyl]sulfamide

Following the procedure described in Example 1a with 2,3′,6-trifluoro-4′-(trifluoromethyl)-4-biphenylamine and purification by Gilson reverse phase HPLC provided the title product. ESMS [M+H]⁺: 371.2.

### Ex 1ee N-[2-chloro-3′-fluoro-4′-(trifluoromethyl)-4-biphenylyl]sulfamide

Following the procedure described in Example 1a with 2-chloro-3′-fluoro-4′-(trifluoromethyl)-4-biphenylamine and purification by Gilson reverse phase HPLC provided the title product. ESMS [M+H]⁺: 369.2.

### Ex 1ff N-[3-chloro-3′-fluoro-4′-(trifluoromethyl)-4-biphenylyl]sulfamide

Following the procedure described in Example 1a with 3-chloro-3′-fluoro-4′-(trifluoromethyl)-4-biphenylamine and purification by Gilson reverse phase HPLC provided the title product. ESMS [M+H]⁺: 369.2.

### Ex 1gg N-[3,3′,5-trifluoro-4′-(trifluoromethyl)-4-biphenylyl]sulfamide

Following the procedure described in Example 1a with 3,3′,5-trifluoro-4′-(trifluoromethyl)-4-biphenylamine and purification by Gilson reverse phase HPLC provided the title product. ESMS [M+H]⁺: 371.2.

### Ex 1hh N-[3′-fluoro-2-(methyloxy)-4′-(trifluoromethyl)-4-biphenylyl]sulfamide

Following the procedure described in Example 1a with 3′-fluoro-2-(methyloxy)-4′-(trifluoromethyl)-4-biphenylamine and purification by Gilson reverse phase HPLC provided the title product. ESMS [M+H]⁺: 365.2.

### Ex 1ii N-[3′-fluoro-3-methyl-4′-(trifluoromethyl)-4-biphenylyl]sulfamide

Following the procedure described in Example 1a with 3′-fluoro-3-methyl-4′-(trifluoromethyl)-4-biphenylamine and purification by Gilson reverse phase HPLC provided the title product. ESMS [M+H]⁺: 349.2.

### Ex 1jj N-[3-ethyl-3′-fluoro-4′-(trifluoromethyl)-4-biphenylyl]sulfamide

Following the procedure described in Example 1a with 3-ethyl-3′-fluoro-4′-(trifluoromethyl)-4-biphenylamine and purification by Gilson reverse phase HPLC provided the title product. ESMS [M+H]⁺: 363.2.

### Ex 1kk N-[3,5-difluoro-4′-(trifluoromethyl)-4-biphenylyl]sulfamide

Following the procedure described in Example 1a with 3,5-difluoro-4′-(trifluoromethyl)-4-biphenylamine and purification by Gilson reverse phase HPLC provided the title product. ESMS [M+H]⁺: 353.2.

### Ex 1ll N-[3′-fluoro-3-(hydroxymethyl)-4′-(trifluoromethyl)-4-biphenylyl]sulfamide

Following the procedure described in Example 1a with 3′-fluoro-3-(hydroxymethyl)-4′-(trifluoromethyl)-4-biphenylamine and purification by Gilson reverse phase HPLC provided the title product. ESMS [M+H]⁺: 365.4.

### Ex 1mm N-[3,3′-difluoro-4′-(trifluoromethyl)-4-biphenylyl]sulfamide

Following the procedure described in Example 1a with 3,3′-difluoro-4′-(trifluoromethyl)-4-biphenylamine provided the title product. ESMS [M+H]⁺: 353.2.

### Ex 1nn N-[3′-nitro-4′-(trifluoromethyl)-4-biphenylyl]sulfamide

Following the procedure described in Example 1a with 3′-nitro-4′-(trifluoromethyl)-4-biphenylamine provided the title product. ESMS [M+H]⁺: 362.2.

### Ex 1oo N-[3-fluoro-3′-nitro-4′-(trifluoromethyl)-4-biphenylyl]sulfamide

Following the procedure described in Example 1a with 3-fluoro-3′-nitro-4′-(trifluoromethyl)-4-biphenylamine provided the title product as an off-white solid (60%). ESMS [M+H]⁺: 380.0.

### PREPARATION 53

### N-[3-fluoro-5-nitro-4′-(trifluoromethyl)-4-biphenylyl]sulfamide

Following the procedure described in Example 1a with 3-fluoro-5-nitro-4′-(trifluoromethyl)-4-biphenylamine provided the title product as a white solid (57%). ESMS [M+H]⁺: 380.2.

### EXAMPLE 2

### N,N-dimethyl-N′-[4′-(trifluoromethyl)-4-biphenylyl]sulfamide

To a solution of 4′-(trifluoromethyl)-4-biphenylamine (0.42 mmol) in dichloromethane (2.0 mL) was sequentially added triethylamine (1.26 mmol) and dimethylaminesulfamoyl chloride (0.63 mmol). The reaction mixture was stirred at room temperature for 18 h and then concentrated in vacuo. Purification by Gilson reverse phase HPLC afforded the title product as a white solid (35%). ESMS [M+H]⁺: 345.2.

### EXAMPLE 3

### N-[3-(methoxy)-4′-(trifluoromethyl)-4-biphenylyl]sulfamide

To a solution of 3-(methoxy)-4-nitro-4′-(trifluoromethyl)biphenyl (1.96 mmol) in acetic acid (10 mL) was added zinc dust (13.7 mmol). The reaction mixure was stirred at room temperature for 2 h and then filtered through Celite and washed with acetic acid (5 mL) and ethanol (5 mL). The filtrate was concentrated in vacuo and the residue was taken up into ethyl acetate (15 mL) and washed with saturated aqueous sodium carbonate solution (10 mL). The organic layer was dried over magnesium sulfate and concentrated in vacuo to give [3-(methoxy)-4′-(trifluoromethyl)-4-biphenylyl]amine as an off-white solid. Following the procedure described in Example 6 with [3-(methoxy)-4′-(trifluoromethyl)-4-biphenylyl]amine provided the title product. ESMS [M-SO₂NH₂]⁺: 267.2. ¹H NMR (DMSO-d₆): δ 8.20 (s, 1H), 7.92 (d, 2H, J=8.2 Hz), 7.80 (d, 2H, J=8.2 Hz), 7.50 (d,1H, J=8.3 Hz), 7.35 (s, 1H), 7.31 (d, 1H, J=8.3 Hz), 7.18 (s, 2H), 3.92 (s, 3H).

### PREPARATION 54

### N-[3-nitro-4′-(trifluoromethyl)-4-biphenylyl]sulfamide

Following the procedure described in Example 1a with [3-nitro-4′-(trifluoromethyl)-4-biphenylyl]amine provided the title product. ESMS [M+H]⁺: 362.0.

### EXAMPLE 4

### N-[3-amino-4′-(trifluoromethyl)-4-biphenylyl]sulfamide

A solution of N-[3-nitro-4′-(trifluoromethyl)-4-biphenylyl]sulfamide (0.73 mmol) in ethanol (10 mL) was reduced in a Parr shaker at 50 psi H₂ with platinum(IV) oxide (0.01 mmol). After 15 min, the reaction mixture was filtered through Celite and concentrated in vacuo. Purification by Gilson reverse phase HPLC afforded the title product as a white solid (81%). ESMS [M+H]⁺: 332.2.

### EXAMPLE 5

### N-[4-[(aminosulfonyl)aminol-4′-(trifluoromethyl)-3-biphenylyl]acetamide

To a solution of N-[3-amino-4′-(trifluoromethyl)-4-biphenylyl]sulfamide (0.28 mmol) in dichloromethane (1.0 mL) was sequentially added triethylamine (0.31 mmol) and acetyl chloride (0.31 mmol). The reaction mixture was stirred at room temperature for 4 h. The reaction mixture was quenched with 0.1N aqueous sodium hydroxide solution (1 mL) and extracted with ethyl acetate (2×5 mL). The combined organic layers were washed with water (5 mL) and brine (5 mL), dried over magnesium sulfate and concentrated in vacuo. Purification by Gilson reverse phase HPLC afforded the title compound as a white solid (5%). ESMS [M+H]⁺: 374.0.

### EXAMPLE 6

### N-[3-hydroxy-4′-(trifluoromethyl)-4-biphenylyl]sulfamide

To a solution of 4-amino-4′-(trifluoromethyl)-3-biphenylol (0.395 mmol) in pyridine (2.0 mL) was added sulfamide (0.435 mmol). The reaction mixture was heated at reflux for 2 h. The reaction mixture was then cooled to room temperature and concentrated in vacuo. Purification of the residue by Gilson reverse phase HPLC afforded the title compound as a light brown solid (22%). ESMS [M+H]⁺: 333.2.

### EXAMPLE 7

### N-[3-(methylamino)-4′-(trifluoromethyl)-4-biphenylyl]sulfamide

A solution of [4-amino-4′-(trifluoromethyl)-3-biphenylyl]methylamine (0.38 mmol) and sulfamide (1.9 mmol) in 1,4-dioxane (1.0 mL) was irradiated in a Personal Chemistry Emrys Optimizer microwave at 150° C. for 10 min. The reaction mixture was concentrated in vacuo. Purification of the residue by Gilson reverse phase HPLC afforded the title compound as a pink solid (5%). ESMS [M+H]⁺: 346.2.

### EXAMPLE 8

### N-methyl-N-[4′-(trifluoromethyl)-4-biphenylyl]sulfamide

To a solution of N-[4′-(trifluoromethyl)-4-biphenylyl]sulfamide (0.36 mmol) in N,N-dimethylformamide (2.0 mL) was added sodium hydride (0.38 mmol). The reaction mixture was stirred at room temperature for 10 min and was treated with methyliodide (0.47 mmol). After 18 h, the reaction mixture was diluted with water (10 mL) and brine (10 mL), and extracted with (3×20 mL) ethyl acetate. The combined organic layers were dried over sodium sulfate and then concentrated in vacuo. Purification of the residue by Gilson reverse phase HPLC afforded the title compound as a white solid (57%). ESMS [M+H]⁺: 331.2.

### EXAMPLE 9

### N-methyl-N′-[4′-(trifluoromethyl)-4-biphenylyl]sulfamide

To a solution of oxalyl chloride (0.59 mmol) in dichloromethane (2.0 mL) was added methylsulfamic acid (0.55 mmol). The reaction mixture was stirred at room temperature for 10 min and was cooled to 0° C. before the addition of 4′-(trifluoromethyl)-4-biphenylamine (0.42 mmol) and pyridine (0.84 mmol) in dichloromethane (2 mL). After stirring at room temperature for 18 h, the reaction mixture was diluted with water (10 mL) and brine (10 mL) and the organics were extracted with (3×20 mL) ethyl acetate. The combined organic layers were dried over sodium sulfate and then concentrated in vacuo. Purification of the residue by Gilson reverse phase HPLC afforded the title compound as a white solid (57%). ESMS [M+H]⁺: 331.2.

### EXAMPLE 10

### N-[4-(2,2-dimethyl-1,3-benzodioxol-5-yl)phenyl]sulfamide

A solution of 3,4—O-isopropylidinebromobenzene (J. Fluorine Chem. 1990, 48, 189-205) (2.62 mmol), [4-(4,4,5,5-tetramethyl-1,3,2-dioxaborolan-2-yl)phenyl]amine (3.14 mmol), and dichloro[1,1′-bis(diphenylphosphino)ferrocene]palladium(II)•dichloromethane adduct (0.26 mmol) in acetonitrile (2.6 mL) and 2M aqueous sodium carbonate solution (2.62 mL) was irradiated in a Personal Chemistry Emrys Optimizer microwave at 150° C. for 8 min. The reaction mixture was filtered through Celite, concentrated in vacuo, and purified by Gilson reverse phase HPLC to afford the intermediate 4-(2,2-dimethyl-1,3-benzodioxol-5-yl)aniline (194 mg). ESMS [M+H]⁺: 242.0.

Approximately half of the 4-(2,2-dimethyl-1,3-benzodioxol-5-yl)aniline was dissolved in dioxane (1 mL) and was treated with sulfamide (3.3 mmol). The reaction mixture was irradiated in a Personal Chemistry Emrys Optimizer microwave at 150° C. for 10 min. The reaction mixture was concentrated in vacuo and the residue was purified by Gilson reverse phase HPLC. Precipitation of the product from water and filtration with a hexanes wash provided the title compound (36 mg). ESMS [M+H]⁺: 321.2.

### EXAMPLE 11

### Ex 11a N-[3′-amino-4′-(trifluoromethyl)-4-biphenylyl]sulfamide

To a stirred solution of N-[3′-nitro-4′-(trifluoromethyl)-4-biphenylyl]sulfamide (0.28 g, 0.78 mMol) in acetic acid (10 mL) was added zinc powder (0.36 g, 5.4 mMol). The suspension was stirred at room temperature for 18 h and then filtered through a pad of Celite, rinsed with acetic acid, and the filtrate was evaporated to dryness in vacuo. The remaining residue was taken up in ethyl acetate, washed sequentially with 1 N aqueous sodium carbonate solution and brine, dried over sodium sulfate, and concentrated in vacuo. Purification by flash chromatography on silica gel (40-60% ethyl acetate/dichloromethane), trituration with hexanes, and drying in vacuo provided the title compound as a white solid (219 mg, 85%). ESMS [M+H]⁺: 301.2.

### Ex 11b N-[3′-amino-3-fluoro-4′-(trifluoromethyl)-4-biphenylyl]sulfamide

Following the procedure described in Example 11a with N-[3-fluoro-3′-nitro-4′-(trifluoromethyl)-4-biphenylyl]sulfamide provided the title compound as a white solid (83%). ESMS [M+H]⁺: 350.0.

### EXAMPLE 12

### N-[3-amino-5-fluoro-4′-(trifluoromethyl)-4-biphenylyl]sulfamide

A solution of N-[3-fluoro-5-nitro-4′-(trifluoromethyl)-4-biphenylyl]sulfamide (0.30 mmol) and 10% palladium on carbon (10% w/w) in methanol was hydrogenated at atmospheric pressure for 2 h at room temperature. The reaction mixture was then filtered through a pad of Celite and concentrated in vacuo. Purification of the residue by Gilson reverse phase HPLC afforded the title product as an orange solid (85%). ESMS [M+H]⁺: 350.2.

### EXAMPLE 13

### Monopolar Spindle Formation Following Application of a KSP Inhibitor of the Invention

Human tumor cells Skov-3 (ovarian) were plated in 96-well plates at densities of 4,000 cells per well, allowed to adhere for 24 hours, and treated with various concentrations of test compounds of the present invention for 24 hours. Cells were fixed in 4% formaldehyde and stained with antitubulin antibodies (subsequently recognized using fluorescently-labeled secondary antibody) and Hoechst dye (which stains DNA).

Compounds of this class were found by visual inspection to cause cell cycle arrest in the prometaphase stage of mitosis, although results varied. Where cell cycle arrest was evident, DNA was condensed and spindle formation had initiated, but arrested cells uniformly displayed monopolar spindles, indicating that there was an inhibition of spindle pole body separation. Microinjection of anti-KSP antibodies also causes mitotic arrest with arrested cells displaying monopolar spindles. Although most of the compounds that inhibited KSP activity biochemically did exhibit cell cycle arrest, for some, cell cycle arrest was not detected.

### EXAMPLE 14

### Inhibition of Cellular Proliferation in Tumor Cell Lines Treated with KSP Inhibitors of the Invention

Cells were plated in 96-well plates at densities from 1000-2500 cells/well of a 96-well plate and allowed to adhere/grow for 24 hours. They were then treated with various concentrations of compounds of the present invention for 48 hours. The time at which compounds are added is considered To. A tetrazolium-based assay using the reagent 3-(4,5-dimethylthiazol-2-yl)-5-(3-carboxymethoxyphenyl)-2-(4-sulfophenyl)-2H-tetrazolium (MTS) (U.S. Pat. No. 5,185,450) (see Promega product catalog #G3580, CellTiter 96® AQᵤₑₒᵤₛ One Solution Cell Proliferation Assay) was used to determine the number of viable cells at T₀ and the number of cells remaining after 48 hours compound exposure. The number of cells remaining after 48 hours was compared to the number of viable cells at the time of drug addition, allowing for calculation of growth inhibition.

The growth over 48 hours of cells in control wells that had been treated with vehicle only (0.25% DMSO) is considered 100% growth and the growth of cells in wells with compounds is compared to this. Compounds of this class were found to inhibit cell proliferation in human ovarian tumor cell lines (SKOV-3), although results varied. Although most of the compounds that inhibited KSP activity biochemically did inhibit cell proliferation, for some compounds inhibition was relatively low or not detected.

A GI₅₀ was calculated by plotting the concentration of compound in μM vs the percentage of cell growth in treated wells. The GI₅₀ calculated for the compounds is the estimated concentration at which growth is inhibited by 50% compared to control, i.e., the concentration at which:
100×[(Treated₄₈-T₀)/(Control₄₈-T₀)]=50.
All concentrations of compounds are tested in duplicate and controls are averaged over 12 wells. A very similar 96-well plate layout and GI₅₀ calculation scheme is used by the National Cancer Institute (see Monks, et al., J. Natl. Cancer Inst. 83:757-766 (1991)). However, the method by which the National Cancer Institute quantitates cell number does not use MTS, but instead employs alternative methods.

Compounds of this class were found to inhibit cell proliferation, although GI₅₀ values varied. GI₅₀ values for the compounds tested ranged from about 140 nM to greater than the highest concentration tested. By this we mean that although most of the compounds that inhibited KSP activity biochemically did inhibit cell proliferation, for some, at the highest concentration tested (generally about 20 μM), cell growth was inhibited less than 50%. Many of the compounds have GI₅₀ values less than 10 μM, and several have GI₅₀ values less than 1 μM. Anti-proliferative compounds that have been successfully applied in the clinic to treatment of cancer (cancer chemotherapeutics) have GI₅₀'s that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation at virtually any concentration may be useful. However, preferably, compounds will have GI₅₀ values of less than 1 mM. More preferably, compounds will have GI₅₀ values of less than 20 μM. Even more preferably, compounds will have GI₅₀ values of less than 10 μM. Further reduction in GI₅₀ values may also be desirable, including compounds with GI₅₀ values of less than 1 μM. Some of the compounds of the invention inhibit cell proliferation with GI₅₀ values below 200 nM.

### EXAMPLE 15

### Calculation of IC₅₀

Measurement of a compound's IC₅₀ for KSP activity uses an ATPase assay. The following solutions are used: Solution 1 consists of 2 mM phosphoenolpyruvate potassium salt (Sigma P-7127), 0.03-1 mM ATP (Sigma A-3377), 1 mM DTT (Sigma D-9779), 10 μM paclitaxel (Sigma T-7402), 250 ppm antifoam 289 (Sigma A-8436), 25 mM Pipes/KOH pH 6.8 (Sigma P6757), 2 mM MgCl₂ (VWR JT400301), and 1 mM EGTA (Sigma E3889). Solution 2 consists of 0.6 mM NADH (Sigma N8129), 0.2 mg/mL BSA (Sigma A7906), pyruvate kinase 7 U/mL, L-lactate dehydrogenase 10 U/mL (Sigma P0294), 50-100 nM KSP motor domain, 200 μg/mL microtubules, 1 mM DTT (Sigma D9779), 10 μM paclitaxel (Sigma T-7402), 250 ppm antifoam 289 (Sigma A-8436), 25 mM Pipes/KOH pH 6.8 (Sigma P6757), 2 mM MgCl₂ (VWR JT4003-01), and 1 mM EGTA (Sigma E3889). Serial dilutions (8-12 two-fold dilutions) of the compound are made in a 96-well microtiter plate (Corning Costar 3695) using Solution 1. Following serial dilution each well has 50 μl of Solution 1. The reaction is started by adding 50 μl of solution 2 to each well. This may be done with a multichannel pipettor either manually or with automated liquid handling devices. The microtiter plate is then transferred to a microplate absorbance reader and multiple absorbance readings at 340 nm are taken for each well in a kinetic mode. The observed rate of change, which is proportional to the ATPase rate, is then plotted as a function of the compound concentration. For a standard IC₅₀ determination the data acquired is fit by the following four parameter equation using a nonlinear fitting program (e.g., Grafit 4):

$$y = \frac{Range}{1 + {( \frac{x}{{IC}_{50}} )}^{s}} + Background$$

where y is the observed rate and x the compound concentration.

The compounds of Figure/Table 1 exhibited a KSP IC₅₀ of 27 μM or less using an ATP concentration of 0.015mM.

Certain intermediate compounds useful for preparing the,compounds of formula I also possess anti-mitotic activity as described above (e.g., as shown by IC₅₀). Such intermediate compounds and their use in the pharmaceutical compositions and the methods described herein also form part of the present invention. Examples of intermediate compounds which possess anti-mitotic activity are 4′-(trifluoromethyl)-4-biphenylamine, [3-fluoro-4′-(trifluoromethyl)-4-biphenylyl]amine, [3-nitro-4′-(trifluoromethyl)-4-biphenylyl]amine, 4′-(trifluoromethyl)-3-biphenylamine, [4-(2,2,4,4-tetrafluoro-4H-1,3-benzodioxin-6-yl)phenyl]amine, 3′-fluoro-4′-(trifluoromethyl)-4-biphenylamine, 4′-[(trifluoromethyl)thio]-4-biphenylamine, 4′-[(trifluoromethyl)sulfonyl]-4-biphenylamine, 4-amino-4′-(trifluoromethyl)-3-biphenylcarbonitrile, 4-(2,2-difluoro-1,3-benzodioxol-5-yl)aniline, 4′-(trifluoromethyl)-3,4-biphenyldiamine, 3-chloro-4′-(trifluoromethyl)-4-biphenylamine, 3,5-difluoro-4′-(trifluoromethyl)-4-biphenylamine, 3-(methyloxy)-4′-(trifluoromethyl)-4-biphenylamine, 4-amino-4′-(trifluoromethyl)-3-biphenylol, 4-(2,2,3,3-tetrafluoro-2,3-dihydro-1,4-benzodioxin-6-yl)aniline, 3-bromo-5-fluoro-4′-(trifluoromethyl)-4-biphenylamine, and [4-amino-4′-(trifluoromethyl)-3-biphenylyl]methylamine.

This application (including the description and claims) may be used as a basis for priority in respect of any subsequent application or claims. The claims of this or such subsequent application may be directed to any feature or combination of features described herein. They may take the form of product, composition, process, or use claims and may include, by way of example and without limitation the following claims.

1. A compound having the structure shown by formula I 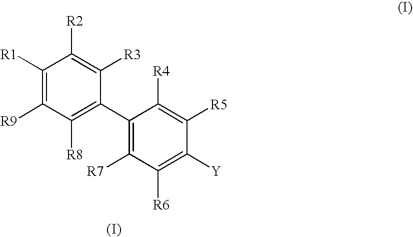
wherein:
one of R1 and R2 is —NR17S(O)ₘNR10R11 and the other is selected from the group consisting of hydrogen, halogen, hydroxy, optionally substituted lower alkoxy, cyano, nitro, optionally substituted lower alkyl, optionally substituted heterocycloalkyl, optionally substituted aryl, optionally substituted heteroaryl, —NR13R14, optionally substituted aminosulfonyl, and optionally substituted aminocarbonyl;
R10 and R11 are independently selected from the group consisting of hydrogen, hydroxy, optionally substituted lower alkoxy, optionally substituted lower alkyl, optionally substituted aralkyl, optionally substituted heteroaralkyl, optionally substituted aryl, and optionally substituted heteroaryl;
R17 is selected from hydrogen and lower alkyl;
m is 1 or 2;
R3, R4, R5, R6, R7, R8, and R9 are independently selected from the group consisting of hydrogen, halogen, hydroxy, optionally substituted lower alkoxy, cyano, nitro, optionally substituted lower alkyl, optionally substituted heterocycloalkyl, optionally substituted aryl, optionally substituted heteroaryl, —NR13R14, optionally substituted aminosulfonyl, and optionally substituted aminocarbonyl;
Y is selected from the group consisting of optionally substituted lower alkyl, halogen, trifluoromethoxy, —S(O)ₙCF₃, —CR15R16CF₃, and —C(X)CF₃;
R15 and R16 taken together with the carbon to which they are attached form a saturated or unsaturated ring having 3 to 6 carbon atoms, optionally containing 1, 2, or 3 heteroatoms selected from nitrogen, sulfur, and oxygen, which ring is optionally substituted with halogen, hydroxy, optionally substituted lower alkoxy, cyano, or optionally substituted lower alkyl;
X is selected from the group consisting of oxygen and sulfur;
n is 0, 1, or 2;
or Y and R5, or Y and R6, taken together with the carbons to which they are attached form a 5 to 7 membered saturated or unsaturated ring optionally containing 1 to 2 heteroatoms selected from oxygen, nitrogen and sulfur, which ring is optionally substituted with halogen, hydroxy, cyano, —NR13R14, optionally substituted lower alkyl, or optionally substituted lower alkoxy, which ring may be aromatic or non-aromatic;
R13 and R14 are independently selected from the group consisting of hydrogen, hydroxy, optionally substituted lower alkoxy, optionally substituted lower alkyl, optionally substituted aryl, and optionally substituted heteroaryl, or R13 and R14 taken together with the nitrogen to which they are attached form a ring having 3 to 7 carbon atoms, optionally containing 1, 2, or 3 heteroatoms selected from nitrogen, sulfur, and oxygen, which ring is optionally substituted with halogen, hydroxy, cyano, optionally substituted lower alkyl, or optionally substituted lower alkoxy;
or a pharmaceutically acceptable derivative or solvate thereof.

2. A compound of claim 1 wherein R1 is —NR17S(O)₂NR10R11.

3. A compound of claim 2 wherein R2 is selected from the group consisting of hydrogen, halogen, hydroxy, optionally substituted lower alkoxy, cyano, nitro, optionally substituted lower alkyl, and —NR13R14.

4. A compound of claim 3 wherein R2 is selected from hydrogen, halogen, hydroxy, methoxy, methyl, cyano and —NH₂.

5. A compound of claim 4 wherein R2 is hydrogen or fluoro.

6. A compound of claim 1 wherein R17 is hydrogen.

7. A compound of claim 1 wherein R10 and R11 are independently selected from hydrogen, hydroxy and optionally substituted lower alkyl.

8. A compound of claim 7 wherein R10 and R11 are independently selected from hydrogen and methyl.

9. A compound of claim 8 wherein R10 and R11 are hydrogen.

10. A compound of claim 1 wherein R3, R4, R7 and R8 are independently selected from the group consisting of hydrogen, halogen, hydroxy, optionally substituted lower alkoxy, cyano, nitro, optionally substituted lower alkyl, and —NR13R14.

11. A compound of claim 10 wherein R3, R4, R7 and R8 are independently selected from hydrogen, halogen, cyano, methoxy, methyl, and —NH₂.

12. A compound of claim 11 wherein R3, R4, R7 and R8 are independently selected from hydrogen and fluoro.

13. A compound of claim 12 wherein R3, R4, R7 and R8 are hydrogen.

14. A compound of claim 1 wherein R9 is selected from the group consisting of hydrogen, halogen, hydroxy, optionally substituted lower alkoxy, cyano, nitro, optionally substituted lower alkyl, and —NR13R14.

15. A compound of claim 14 wherein R9 is selected from the group consisting of hydrogen, halogen, methyl and —NH₂.

16. A compound of claim 15 wherein R9 is hydrogen or fluoro.

17. A compound of claim 16 wherein R9 is hydrogen.

18. A compound of claim 1 wherein Y is halogen, trifluoromethoxy, S(O)ₙCF₃, optionally substituted lower alkyl, or —CR15R16CF₃.

19. A compound of claim 18, wherein Y is fluoro, chloro, bromo, trifluoromethoxy, S(O)₂CF₃, S(O)CF₃, isobutyl, t-butyl, isopropyl, or trifluoromethyl.

20. A compound of claim 19, wherein Y is trifluoromethyl, S(O)₂CF₃, isopropyl, or t-butyl.

21. A compound of claim 1 wherein R5 and R6 are independently selected from the group consisting of hydrogen, halogen, hydroxy, optionally substituted lower alkoxy, cyano, nitro, optionally substituted lower alkyl, and —NR13R14.

22. A compound of claim 21 wherein R5 and R6 are independently selected from hydrogen, halogen, methyl, nitro, trifluoromethyl, and —NH₂.

23. A compound of claim 22 wherein R5 and R6 are independently selected from hydrogen and fluoro.

24. A compound of claim 1, wherein Y and R5, or Y and R6, taken together with the carbons to which they are attached form an optionally substituted 5 or 6 membered saturated or unsaturated ring containing 1 to 2 heteroatoms selected from oxygen, nitrogen and sulfur, which ring may be aromatic or non-aromatic.

25. A compound of claim 24 wherein the 5 or 6 membered ring contains 1 to 2 atoms selected from oxygen and sulfur and is substituted with one or more halogen, lower alkyl, and/or lower haloalkyl groups.

26. A compound of claim 25 wherein the ring is substituted with one or more fluoro, methyl and/or trifluoromethyl groups.

27. A compound selected from:
N-[4′-(trifluoromethyl)-4-biphenylyl]sulfamide,
N-[3-fluoro-4′-(trifluoromethyl)-4-biphenylyl]sulfamide,
N,N-dimethyl-N-[4′-(trifluoromethyl)-4-biphenylyl]sulfamide,
N-[4′-(trifluoromethyl)-3-biphenylyl]sulfamide,
N-[3′-fluoro-4′-(trifluoromethyl)-4-biphenylyl]sulfamide,
N-[4-(2,2-difluoro-1,3-benzodioxol-5-yl)phenyl]sulfamide,
N-[4-(1,3-benzodioxol-5-yl)phenyl]sulfamide,
N-[4-(1,3-benzodioxol-5-yl)-2-fluorophenyl]sulfamide,
N-[3-cyano-4′-(trifluoromethyl)-4-biphenylyl]sulfamide,
N-[2′-cyano-3-fluoro-4′-(trifluoromethyl)-4-biphenylyl]sulfamide,
N-[3-(methoxy)-4′-(trifluoromethyl)-4-biphenylyl]sulfamide,
N-[3-amino-4′-(trifluoromethyl)-4-biphenylyl]sulfamide,
N-[4-[(aminosulfonyl)amino]-4′-(trifluoromethyl)-3-biphenylyl]acetamide,
N-[4-(2,2,4,4-tetrafluoro-4H-1,3-benzodioxin-6-yl)phenyl]sulfamide,
N-[2-cyano-4′-(trifluoromethyl)-4-biphenylyl]sulfamide,
N-{4′-[(trifluoromethyl)sulfinyl]-4-biphenylyl}sulfamide,
N-{4′-[(trifluoromethyl)sulfonyl]-4-biphenylyl}sulfamide,
N-[3,3′-difluoro-4′-(trifluoromethyl)-4-biphenylyl]sulfamide,
N-[3′-nitro-4′-(trifluoromethyl)-4-biphenylyl]sulfamide,
N-[3′-amino-4′-(trifluoromethyl)-4-biphenylyl]sulfamide,
N-[4-(2,2,3,3-tetrafluoro-2,3-dihydro-1,4-benzodioxin-6-yl)phenyl]sulfamide,
N-[3-hydroxy-4′-(trifluoromethyl)-4-biphenylyl]sulfamide,
N-[3-fluoro-3′-nitro-4′-(trifluoromethyl)-4-biphenylyl]sulfamide,
N-[3′-amino-3-fluoro-4′-(trifluoromethyl)-4-biphenylyl]sulfamide,
N-methyl-N-[4′-(trifluoromethyl)-4-biphenylyl]sulfamide,
N-[3-ethyl-3′-fluoro-4′-(trifluoromethyl)-4-biphenylyl]sulfamide,
N-[2-methyl-4′-(trifluoromethyl)-4-biphenylyl]sulfamide,
N-[3-methyl-4′-(trifluoromethyl)-4-biphenylyl]sulfamide,
N-[2-fluoro-4′-(trifluoromethyl)-4-biphenylyl]sulfamide,
N-[2,5-difluoro-4′-(trifluoromethyl)-4-biphenylyl]sulfamide,
N-[2,6-difluoro-4′-(trifluoromethyl)-4-biphenylyl]sulfamide,
N-[2-chloro-4′-(trifluoromethyl)-4-biphenylyl]sulfamide,
N-[3-chloro-4′-(trifluoromethyl)-4-biphenylyl]sulfamide,
N-[2-(methyloxy)-4′-(trifluoromethyl)-4-biphenylyl]sulfamide,
N-[3′-fluoro-2-methyl-4′-(trifluoromethyl)-4-biphenylyl]sulfamide,
N-[2,3′-difluoro-4′-(trifluoromethyl)-4-biphenylyl]sulfamide,
N-[2,3′,5-trifluoro-4′-(trifluoromethyl)-4-biphenylyl]sulfamide,
N-[2,3′,6-trifluoro-4′-(trifluoromethyl)-4-biphenylyl]sulfamide,
N-[2-chloro-3′-fluoro-4′-(trifluoromethyl)-4-biphenylyl]sulfamide,
N-[3-chloro-3′-fluoro-4′-(trifluoromethyl)-4-biphenylyl]sulfamide,
N-[3,3′,5-trifluoro-4′-(trifluoromethyl)-4-biphenylyl]sulfamide,
N-[3′-fluoro-2-(methyloxy)-4′-(trifluoromethyl)-4-biphenylyl]sulfamide,
N-[3,5-difluoro-4′-(trifluoromethyl)-4-biphenylyl]sulfamide,
N-[3′-fluoro-3-methyl-4′-(trifluoromethyl)-4-biphenylyl]sulfamide,
N-methyl-N′-[4′-(trifluoromethyl)-4-biphenylyl]sulfamide,
N-[2-(hydroxymethyl)-4′-(trifluoromethyl)-4-biphenylyl]sulfamide,
N-[3-(hydroxymethyl)-4′-(trifluoromethyl)-4-biphenylyl]sulfamide,
N-[3′-fluoro-3-(hydroxymethyl)-4′-(trifluoromethyl)-4-biphenylyl]sulfamide,
N-[3-(methylamino)-4′-(trifluoromethyl)-4-biphenylyl]sulfamide,
N-[4-(2,2-dimethyl-1 ,3-benzodioxol-5-yl)phenyl]sulfamide,
N-[3-bromo-5-fluoro-4′-(trifluoromethyl)-4-biphenylyl]sulfamide,
N-[3-amino-5-fluoro-4′-(trifluoromethyl)-4-biphenylyl]sulfamide, and
N-[3-[(1-methylethyl)amino]-4′-(trifluoromethyl)-4-biphenylyl]sulfamide.

28. A composition comprising a pharmaceutically acceptable excipient and the compound or derivative or solvate thereof of claim 1.

29. A method of modulating KSP kinesin activity or of inhibiting KSP which comprises contacting said kinesin with an effective amount of the compound or derivative or solvate thereof of claim 1.

30. A method for the treatment of a disease of proliferating cells comprising administering to a subject in need thereof the compound or derivative or solvate thereof of claim 1.

31. A method according to claim 30 wherein said disease is selected from the group consisting of cancer, hyperplasias, restenosis, cardiac hypertrophy, immune disorders, fungal disorders and inflammation.
